(19) United States
(12) Patent Application Publication
(10) Pub. No.: US 2007/0248561 A1
(43) Pub. Date: Oct. 25, 2007

(54) Water-soluble polymers and their use in cosmetic and pharmaceutical preparations

(75) Inventors: Robert Milbradt, Wiesbaden (DE); Carina Mildner, Frankfurt am Main (DE)

Correspondence Address: CLARIANT CORPORATION;INTELLECTUAL PROPERTY DEPARTMENT, 4000 MONROE ROAD, CHARLOTTE, NC, 28205, US

(73) Assignee: Clariant International Ltd

(21) Appl. No.: 11/788,452

(22) Filed: Apr. 20, 2007

(30) Foreign Application Priority Data
Apr. 21, 2006 (DE) 102006018523.4

(51) Int. Cl. A61K 8/81

(52) U.S. Cl. 424/70.16

(57) ABSTRACT

A description is given of cosmetic and pharmaceutical preparations comprising
I) one or more water-soluble noncrosslinked copolymers containing
A) one or more structural units of the formula (1)

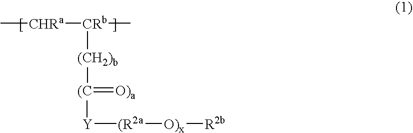

and
B) one or more structural units of the formula (2)

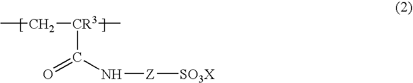

and
II) one or more water-soluble or water-swellable crosslinked or noncrosslinked copolymeric or homopolymeric thickeners.

The present invention relates to a mixture of noncrosslinked polymers, which have been prepared by copolymerizing macromonomers and comonomers based on acryloyldimethyltaurine and/or salts thereof, and at least one further water-soluble or water-swellable polymeric thickener, and to the use of the polymer mixture in cosmetic and pharmaceutical compositions.

Consumer requirements and cosmetic product rheology are closely interlinked. Thus, for example, the visual appearance of a cream or lotion is influenced by its viscosity. The sensorial properties, such as consistency or spreadability, determine the individual profile of a cosmetic product. The effectiveness of active substances (e.g., sunscreen filters) and the storage stability of the formulation are also closely related to the rheological properties of the products.

In the cosmetics sector a leading part is played by polyelectrolytes as thickeners and gel formers. State of the art are, in particular, thickeners based on poly(meth)acrylic acid and the water-soluble copolymers thereof. The diversity of the possible structures, and the diverse possibilities for use that are associated therewith, are manifested not least in a multiplicity of patents filed worldwide since the mid-1970s.

A substantial drawback of thickeners based on poly(meth)acrylic acid is the heavy pH dependency of the thickening performance. Thus, in general, a sufficiently high viscosity is developed only when the pH of the formulation is adjusted to a level of more than 6, i.e., the poly(meth)acrylic acid is in neutralized form. Furthermore, the corresponding compositions are sensitive to UV radiation and also to shearing, and they also impart a sticky feeling on the skin. The handling of such thickeners is also problematic. Since the thickeners are generally in an acidic form, formulation requires an additional neutralizing step.

In the 1990s, innovative thickeners based on crosslinked and neutralized polyacryloyldimethyltaurates were introduced into the market (EP-B-0 815 828, EP-B-0 815 844, EP-B-0 815 845 and EP-A-0 850 642). In the form both of the preneutralized homopolymer and of the corresponding copolymer (Aristoflexe AVC, Clariant GmbH), these thickeners are superior in many respects to the poly(meth)acrylate types. For example, acryloyldimethyltaurate-based thickener systems display outstanding properties in pH ranges below a pH of 6, in other words in a pH range in which it is no longer possible to operate with conventional poly(meth)acrylate thickeners. High UV stability and shearing stability, outstanding viscoelastic properties, great ease of processing, and a favorable toxicological profile of the principal monomer make acryloyidimethyltaurate-based thickener systems modern, new candidates with a high potential for the future.

Over the course of recent years, a further thickener concept has become established on the market. In this case, by hydrophobic modification of the conventional poly(meth)acrylates, access has been gained to polymers which have not only thickening but also emulsifying/dispersing properties. Examples of commercial hydrophobically modified poly(meth)acrylates are Pemulen® TR-1 and TR-2 from BF Goodrich and Aculyn® 22 from Rohm and Haas. Since these hydrophobically modified polymers are based on (meth)acrylic acid, they also possess the abovementioned drawbacks of the poly(meth)acrylates.

EP 1 069 142 describes hydrophobically modified copolymers based on acryloyidimethyltaurine and/or its salts, and their use as a thickener, dispersant, suspension agent, emulsifier, stabilizer, and consistency agent. Whereas the noncrosslinked copolymers of this polymer class exhibit little thickening capacity and have a tendency toward stringing, the crosslinked types are outstanding thickeners. A drawback is that the viscosity of the formulations thickened with crosslinked acryloyidimethyltaurine copolymers falls sharply in the presence of electrolytes, and the gel structure breaks down.

The object was to provide substances for cosmetic, pharmaceutical, and dermatological preparations that have good thickening and consistency-imparting properties and at the same time high electrolyte stability, that are highly compatible with aqueous systems and with oil systems, and also with cosmetic product ingredients, such as surfactants, that have a clear visual appearance, are easy to process, and are compatible with active substances (e.g., sunscreen filters), exhibit temperature and storage stability, but are also skin-friendly and toxicologically unobjectionable.

It has surprisingly been found that this object is achieved by a combination of noncrosslinked copolymers based on acryloyldimethyltaurine and/or its salts, with at least one further water-soluble or water-swellable polymeric thickener.

Through the use of a mixture of noncrosslinked copolymers based on acryloyldimethyltaurine and/or its salts, preferably comprising the corresponding noncrosslinked hydrophobically modified copolymers, with at least one further water-soluble or water-swellable polymeric thickener it is possible in aqueous systems in particular to produce electrolyte-stable gels having good pH stability, good skin sensorial properties, little stickiness, and stable consistency. The copolymer mixture is outstandingly suitable for use as a consistency agent and thickener, especially for hairstyling compositions.

The invention provides cosmetic or pharmaceutical preparations comprising

### I) one or more water-soluble noncrosslinked copolymers containing

### A) one or more structural units of the formula (1)

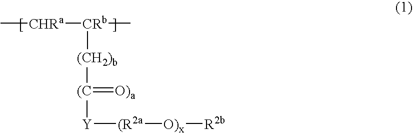

in which

### R^{a} is H or CH₃;

### R^{b} is H or CH₃;

a is 0 or 1;
b is 0 or 1;

### Y is O, S, PH or NH;

### R^{2a} is a linear or branched (C₂-C₄)-alkylene group;

x is an integer between 1 and 500; and

### R^{2b} is hydrogen or a saturated or mono- or polyunsaturated linear or branched aliphatic, cycloaliphatic or aromatic (C₁-C₃₀)-hydrocarbon radical, and

13. one or more structural units of the formula (2)

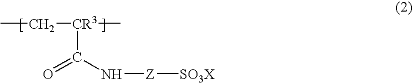

in which

### R³ is hydrogen, methyl or ethyl,

### Z is (C₁-C₈)-alkylene, and

X is hydrogen, lithium, sodium, potassium, magnesium, calcium, ammonium, monoalkylammonium, dialkylammonium, trialkylammonium or tetraalkylammonium, the alkyl substituents of the ammonium ions being independently of one another (C₁-C₂₂)-alkyl radicals which may be occupied by 0 to 3 hydroxyalkyl groups whose alkyl chain length can vary in a range from C₂ to C₁₀, or else X is singly to triply ethoxylated ammonium compounds having the same degree or different degrees of ethoxylation, it also being possible for structural units of the formula (2) with different Xs to be present in the noncrosslinked copolymers, and

### II) one or more water-soluble or water-swellable crosslinked or noncrosslinked copolymeric or homopolymeric thickeners.

The structural units of the formula (1) are preferably structural units originating from macromonomers.

Macromonomers for the purposes of the present invention are polymerizable chemical compounds which carry at least one olefinic double bond and which in a polymerization reaction lead to structural units of the formula (1), the variable x in the repeating unit (R^{2a}—O)ₓ having on average a value greater than 1.

Within one structural unit of the formula (1) it is also possible for (R^{2a}—O) to take on different definitions.

In one preferred embodiment of the invention R^{a}, R^{b}, a, and b in the structural unit of the formula (1) are selected from the following combinations:

### R^{a}=R^{b}=H and a=b=0;

### R^{a}=R^{b}=H, a=0 and b=1;

### R^{a}=R^{b}=H, a=1 and b=0; or

### R^{a}=H, R^{b}=CH₃, a=1 and b=0.

In one particularly preferred embodiment of the invention R^{a}, R^{b}, a, and b in the structural unit of the formula (1) are selected from the following combinations:

### R^{a}=R^{b}=H, a=1 and b=0 or

### R^{a}=H, R^{b}=CH₃, a=1 and b=0.

In a further preferred embodiment of the invention, in the structural unit of the formula (1),
R^{2a} is an ethylene or propylene radical, preferably an ethylene radical,
x is a number between 3 and 50, preferably between 6 and 30, and
R^{2b} is a saturated or a mono- or polyunsaturated aliphatic or cycloaliphatic hydrocarbon radical.

In a further preferred embodiment of the invention R^{2b} in the structural unit of the formula (1) is a (C₆-C₂₂)-hydrocarbon radical, preferably a (C₁₂-C₁₈)-hydrocarbon radical. With particular preference this hydrocarbon radical is an alkyl or a mono- or polyunsaturated alkenyl radical, preferably an alkyl radical.

In a further preferred embodiment of the invention R^{2b} in the structural unit of the formula (1) is a radical selected from stearyl, lauryl, cocoyl, undecyl, behenyl, cetearyl, cetyl, and myristyl, and preferably a radical selected from stearyl, lauryl, cetyl, and myristyl.

In a further preferred embodiment of the invention, in the structural unit of the formula (2), R³ is H, Z is —C(CH₃)₂—CH₂—, and X is hydrogen, sodium, potassium or ammonium, preferably H or ammonium, and in the noncrosslinked copolymers there may also be structural units of the formula (2) having different Xs.

In a further preferred embodiment of the invention the degree of neutralization of the structural unit of the formula (2) is 70 to 100 mol %, preferably 80 to 100 mol %, and more preferably 80 to 99 mol %.

In a further preferred embodiment of the invention the molar fractions of the structural unit of the formula (1) and of the structural unit of the formula (2) in the copolymer of component I) are in each case from 0.1 to 99.9 mol %.

In one particularly preferred embodiment of the invention the fraction of the structural unit of the formula (1) in the copolymer of component I) is from 50.1 to 99.9 mol %, preferably from 70 to 95 mol %, and more preferably from 80 to 90 mol %.

In another particularly preferred embodiment of the invention the fraction of the structural unit of the formula (1) in the copolymer of component I) is from 0.1 to 50 mol %, preferably from 5 to 25 mol %, and more preferably from 6 to 15 mol %.

Additionally preferred cosmetic or pharmaceutical preparations of the invention are those comprising
I) one or more water-soluble noncrosslinked copolymers preparable by free-radical copolymerization of

### A) one or more macromonomers of the formula (1a)

R¹¹—Y—(R²¹—O)ₓ—R³¹ (1a)

in which R¹¹ is a vinyl, allyl, acryloyl or methacryloyl radical; R²¹ is (C₂-C₄)-alkylene; x is an integer between 1 and 500; Y=O, S, PH or NH; and R³¹ is hydrogen or a saturated or unsaturated linear or branched aliphatic, cycloaliphatic or aromatic (C₁-C₃₀)-hydrocarbon radical, and

a. one or more monomers of the formula (2a)

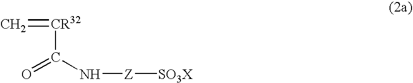

in which R³² is hydrogen, methyl or ethyl, Z is (C₁-C₈)-alkylene, and X is hydrogen, lithium, sodium, potassium, magnesium, calcium, ammonium, monoalkylammonium, dialkylammonium, trialkylammonium or tetraalkylammonium, the alkyl substituents of the ammonium ions being independently of one another (C₁-C₂₂)-alkyl radicals which may be occupied by 0 to 3 hydroxyalkyl groups whose alkyl chain length can vary in a range from C₂ to C₁₀, or else X is singly to triply ethoxylated ammonium compounds having different degrees of ethoxylation, it also being possible for structural units originating from the monomers of the formula (2a) with different Xs to be present in the noncrosslinked copolymers and
II) one or more water-soluble or water-swellable crosslinked or noncrosslinked copolymeric or homopolymeric thickeners.

In the compounds of the formula (1a) R¹¹ is preferably an acryloyl or methacryloyl radical.

In the compounds of the formula (1a) R²¹ is preferably an ethylene or propylene radical.

In the compounds of the formula (1a) x is preferably a number between 3 and 50, more preferably a number between 6 and 30.

In the compounds of the formula (1a) R³¹ is preferably aliphatic or cycloaliphatic hydrocarbons, which may be saturated or unsaturated, more preferably a (C₆-C₂₂)-hydrocarbon radical, with particular preference a (C₁₂-C₁₈)-hydrocarbon radical. With extraordinary preference R³¹ is an alkyl or a mono- or polyunsaturated alkenyl radical, of these preferably, in turn, the alkyl radical. With very extraordinary preference R³¹ is a radical selected from stearyl, lauryl, cocoyl, undecyl, behenyl, cetearyl, cetyl and myristyl, and of these preferably, in turn, a radical selected from stearyl, lauryl, cetyl and myristyl.

In the compounds of the formula (2a) R³² is preferably H, Z is preferably —C(CH₃)₂—CH₂—, and X is preferably hydrogen, sodium, potassium or ammonium, more preferably hydrogen or ammonium, it also being possible in the noncrosslinked copolymers for there to be structural units of the formula (2a) with different Xs.

Preferably the degree of neutralization of the structural unit originating from the monomers of the formula (2a) is from 70 to 100 mol %, preferably from 80 to 100 mol %, and more preferably from 80 to 99 mol %.

In a further preferred embodiment of the invention the molar fractions of the structural unit originating from the macromonomers of the formula (1a) and of the structural unit originating from the monomers of the formula (2a) in the copolymer of component I) are in each case from 0.1 to 99.9 mol %.

In one particularly preferred embodiment of the invention the fraction of the structural unit originating from the macromonomers of the formula (1a) in the copolymer of component I) is from 50.1 to 99.9 mol %, preferably from 70 to 95 mol %, and more preferably from 80 to 90 mol %.

In another particularly preferred embodiment of the invention the fraction of the structural unit originating from the macromonomers of the formula (1a) in the copolymer of component I) is from 0.1 to 50 mol %, preferably from 5 to 25 mol %, and more preferably from 6 to 15 mol %.

Preferred water-soluble or water-swellable crosslinked or noncrosslinked copolymeric or homopolymeric thickeners of component II) are selected from
a) polymers based on methacrylic acid or acrylic acid and modified (meth)acrylic acid, preferably crosslinked polymers of acrylic acid of the kind available under the trade names Carbopol 980, 981, 954, 2984 and 5984 (CTFA name: Carbomer) or Synthalen M and Synthalen K, copolymers of (meth)acrylic acid and polyalkylene polyether, and hydrophobically modified poly(meth)acrylates, examples being the copolymers available as Pemulen® TR-1 and TR-2 from BF Goodrich, Carbopol® ETD 2020 from BF Goodrich (Acrylate/C10⁻³⁰ Alkyl Acrylate Polymer), Aculyn® 22 from Rohm and Haas (Acrylates/Steareth-20 Methacrylate Copolymer), Aculyn® 28 from Rohm and Haas (Acrylates/Beheneth-25 Methacrylate Copolymer), Synthalen® W 2000 from 3V Sigma (Acrylate/Palmeth-25 Acrylate Copolymer), Structure® 3001 from National Starch (Acrylates/Ceteth-20 Itaconate Copolymer),
b) homopolymers of dimethylaminoethyl(meth)acrylates, quaternized with methyl chloride, as obtainable under the trade names Salcare® 95 and Salcare® 96 from Ciba,
c) copolymers of dimethylaminoethyl(meth)acrylate, quaternized with methyl chloride and acrylamide, as obtainable under the trade names Salcare® SC92 or PAS 5194,
d) crosslinked copolymers of vinyl isodecanoate and (meth)acrylic acid, as availabe under the trade name Stabylene 30,
e) polyvinyl alcohols,
f) polyvinyl methyl ethers,
g) polyacrylamides,
h) polyvinylamides,
i) polyvinylpyrrolidone,
j) poly(meth)acrylic acids, poly(meth)acrylic esters, and other poly(meth)acrylic acid derivatives,
k) polyethylene oxides,
l) copolymers of maleic anhydride and vinyl methyl ether,
m) polysulfonic acids, preferably copolymers based on acrylamidoalkylsulfonic acid and/or salts thereof and one or more comonomers selected from cyclic N-vinylcarboxamides and linear N-vinylcarboxamides, or else hydrophobically modified crosslinked acrylamidoalkylsulfonic acid copolymers (for example as described in DE 10 059 826),
n) crosslinked homopolymers of acrylamidoalkylsulfonic acid and/or salts thereof,
o) copolymers of acrylamidoalkylsulfonic acid and/or salts thereof, and comonomers selected from acrylamide, hydroxyethyl(meth)acrylate and cationically modified (meth)acrylates, and
p) natural and modified natural polymers based on polysaccharides, preferably cellulose ethers, cellulose derivatives, carboxymethylcellulose, hydroxyethylcellulose, gelatin, starch and starch derivatives, sodium alginates, xanthan, guar and guar derivatives, scleroglucan, tragacanth or dextrin derivatives, especially dextrin esters.

Particularly preferred water-soluble or water-swellable crosslinked or noncrosslinked copolymeric or homopolymeric thickeners of component II) are selected from the abovementioned groups a), g), j), m), n), o) and p).

In one further preferred embodiment of the invention the weight fraction of crosslinking comonomers, based on the total mass of the polymers of component II), is from 0% to 20% by weight.

In a further preferred embodiment of the invention the cosmetic or pharmaceutical preparations comprise one or more water-soluble noncrosslinked copolymers of component I) based on acryloyidimethyltaurine and/or its salts, preferably corresponding noncrosslinked hydrophobically modified copolymers of component I), and one or more water-soluble or water-swellable crosslinked copolymeric thickeners of component II) based on acryloyldimethyltaurine and/or its salts, preferably corresponding crosslinked hydrophobically modified copolymeric thickeners of component II), the noncrosslinked copolymers of component I) and the crosslinked copolymers of component II) in each case being preparable by free-radical copolymerization of

### A) one or more macromonomers of the formula (1a)

R¹¹—Y— (R²¹—O)ₓ—R³¹ (1a)

in which R¹¹ is a vinyl, allyl, acryloyl or methacryloyl radical; R²¹ is (C₂-C₄)-alkylene; x is an integer between 1 and 500; Y=O, S, PH or NH; and R³¹ is hydrogen or a saturated or unsaturated linear or branched aliphatic, cycloaliphatic or aromatic (C₁-C₃₀)-hydrocarbon radical, and

### B) one or more comonomers of the formula (2a)

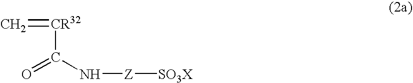

in which R³² may be hydrogen, methyl or ethyl, Z may be (C₁-C₈)-alkylene, and X may be a hydrogen, an ammonium, alkali metal or alkaline earth metal ion, it also being possible for structural units originating from the monomers of the formula (2a) with different Xs to be present in the noncrosslinked copolymers of component I) and the crosslinked copolymers of component II)
and only in the case of the crosslinked copolymers of component II)

### B) additionally one or more crosslinkers.

This way of writing means in the context of the present invention that the copolymers of component I) are preparable by free-radical copolymerization of the monomers of the formulae (1a) and (2a), and the copolymers of component II) are preparable by free-radical copolymerization of the monomers of the formulae (1a) and (2a) and additionally one or more crosslinkers.

The crosslinkers of group C) are monomers having two or more double bonds.

In the compounds of the formula (1a) R¹¹ is preferably an acryloyl or methacryloyl radical.

In the compounds of the formula (1a) R²¹ is preferably an ethylene or propylene radical.

In the compounds of the formula (1a) x is preferably a number between 3 and 50, more preferably a number between 6 and 30.

In the compounds of the formula (1a) R³¹ is preferably aliphatic or cycloaliphatic hydrocarbons, which may be saturated or unsaturated, more preferably a (C₆-C₂₂)-hydrocarbon radical, with particular preference a (C₁₂-C₁₈)-hydrocarbon radical. With extraordinary preference R³¹ is an alkyl or a mono- or polyunsaturated alkenyl radical, of these preferably, in turn, the alkyl radical. With very extraordinary preference R³¹ is a radical selected from stearyl, lauryl, cocoyl, undecyl, behenyl, cetearyl, cetyl and myristyl, and of these preferably, in turn, a radical selected from stearyl, lauryl, cetyl and myristyl.

In the compounds of the formula (2a) R³² is preferably H, Z is preferably —C(CH₃)₂—CH₂—, and X is preferably hydrogen, sodium, potassium or ammonium, more preferably hydrogen or ammonium, it also being possible in the noncrosslinked copolymers of component I) and in the crosslinked copolymers of component II) for there to be structural units of the formula (2a) with different Xs.

Preferably the degree of neutralization of the structural unit originating from the monomers of the formula (2a) is from 70 to 100 mol %, preferably from 80 to 100 mol %, and more preferably from 80 to 99 mol %.

In a further preferred embodiment of the invention the molar fractions of the structural unit originating from the macromonomers of the formula (1a) and of the structural unit originating from the monomers of the formula (2a) in the copolymer of component I) are in each case from 0.1 to 99.9 mol %.

In a further preferred embodiment of the invention the molar fractions of the structural unit originating from the macromonomers of the formula (1a) and of the structural unit originating from the monomers of the formula (2a) in the copolymer of component II) are in each case from 0.1 to 99.85 mol %, and the molar fraction of the structural unit originating from the one or more crosslinkers is from 0.05 to 8 mol %.

In one particularly preferred embodiment of the invention the fraction of the structural unit originating from the macromonomers of the formula (1a) in the copolymer of component I) is from 50.1 to 99.9 mol %, preferably from 70 to 95 mol %, and more preferably from 80 to 90 mol %.

In one further particularly preferred embodiment of the invention the fraction of the structural unit originating from the macromonomers of the formula (1a) in the copolymer of component II) is from 50.1 to 99.85 mol %, preferably from 70 to 95 mol %, and more preferably from 80 to 90 mol %, and the molar fraction of the structural unit originating from the one or more crosslinkers is from 0.05 to 8 mol %.

In another particularly preferred embodiment of the invention the fraction of the structural unit originating from the macromonomers of the formula (1a) in the copolymer of component I) is from 0.1 to 50 mol %, preferably from 5 to 25 mol %, and more preferably from 6 to 15 mol %.

In one further particularly preferred embodiment of the invention the fraction of the structural unit originating from the macromonomers of the formula (1a) in the copolymer of component II) is from 0.1 to 50 mol %, preferably from 5 to 25 mol %, and more preferably from 6 to 15 mol %, and the molar fraction of the structural unit originating from the one or more crosslinkers is from 0.05 to 8 mol %.

Additionally preferred preparations of the invention are those in which the water-soluble or water-swellable crosslinked or noncrosslinked copolymeric or homopolymeric thickener or thickeners of component II) are selected from copolymers composed essentially of

a1) 1% to 50% by weight of the structural repeating unit of the formula (3)

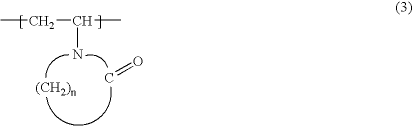

where n is an integer from 2 to 9 or
a2) 1% to 50% by weight of the structural repeating unit of the formula (4)

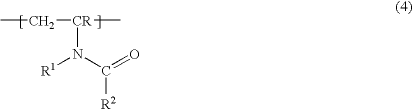

where R, R¹ and R² can be identical or different and are hydrogen or a linear or branched alkyl or alkenyl group having in each case 1 to 30, preferably 1 to 20, in particular 1 to 12, carbon atoms or
a3) 1% to 50% by weight of a mixture of the structural repeating unit of the formula (3) and the structural repeating unit of the formula (4), and
b) 49.99% to 98.99% by weight of the structural repeating unit of the formula (2)

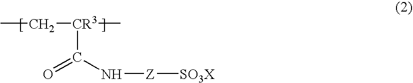

in which

### R³ is hydrogen, methyl or ethyl,

### Z is (C₁-C₈)-alkylene, and

X is hydrogen, lithium, sodium, potassium, magnesium, calcium, ammonium, monoalkylammonium, dialkylammonium, trialkylammonium or tetraalkylammonium, the alkyl substituents of the ammonium ions being independently of one another (C₁-C₂₂)-alkyl radicals which may be occupied by 0 to 3 hydroxyalkyl groups whose alkyl chain length can vary in a range from C₂ to C₁₀, or else X is singly to triply ethoxylated ammonium compounds having the same degree or different degrees of ethoxylation, it also being possible for structural units of the formula (2) with different Xs to be present in the copolymers, and
c) 0% to 8% by weight of crosslinking structures originating from monomers having at least two olefinic double bonds.

In a further preferred embodiment of the invention the water-soluble or water-swellable crosslinked or noncrosslinked copolymeric or homopolymeric thickener or thickeners of component II) is or are selected from crosslinked polymers comprising crosslinking structures originating from methylenebisacrylamide; methylenebismethacrylamide; esters of unsaturated monocarboxylic and polycarboxylic acids with polyols, preferably di-acrylates and tri-acrylates and -methacrylates, more preferably butanediol and ethylene glycol diacrylate and methacrylate, trimethylolpropane triacrylate (TMPTA) and trimethylolpropane trimethacrylate (TMPTMA); allyl compounds, preferably allyl(meth)acrylate, triallyl cyanurate, diallyl maleate, polyallyl esters, tetraallyloxyethane, triallylamine, tetraallylethylenediamine; allyl esters of phosphoric acid; and/or vinylphosphonic acid derivatives, preferably trimethylolpropane triacrylate (TMPTA).

Further preferred preparations of the invention comprise as component II) one or more crosslinked homopolymers composed in random distribution of 90% to 99.99% by weight of structural units originating from monomers of the formula (2a) and of 0.01% to 10% by weight of crosslinking structures originating from monomers having at least two olefinic double bonds. Preferred crosslinkers in this context are methylenebisacrylamide; methylenebismethacrylamide; esters of unsaturated monocarboxylic and polycarboxylic acids with polyols, preferably di-acrylates and tri-acrylates and -methacrylates, more preferably butanediol and ethylene glycol diacrylate and methacrylate, trimethylolpropane triacrylate (TMPTA) and trimethylolpropane trimethacrylate (TMPTMA); allyl compounds, preferably allyl (meth)acrylate, triallyl cyanurate, diallyl maleate, polyallyl esters, tetraallyloxyethane, triallylamine, tetraallylethylenediamine; allyl esters of phosphoric acid; and/or vinylphosphonic acid derivatives. With particular preference the crosslinking structures originate from trimethylolpropane triacrylate (TMPTA).

In one particularly preferred embodiment of the invention the water-soluble or water-swellable crosslinked or noncrosslinked copolymeric or homopolymeric thickener or thickeners of component II) is or are selected from crosslinked polymers comprising crosslinking structures originating from trimethylolpropane triacrylate.

In a further preferred embodiment of the invention the cosmetic, pharmaceutical or dermatological preparations comprise one or more noncrosslinked copolymers of component I) and one or more crosslinked polymers of component II), in each case preparable by free-radical copolymerization of

### A) one or more macromonomers of the formula (5)

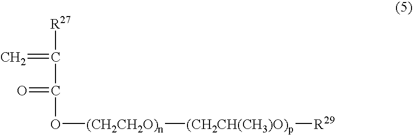

where R²⁷ is hydrogen or methyl, R²⁹ is a linear or branched alkyl group having 7 to 22, preferably 8 to 18 and more preferably 12 to 18 carbon atoms, the indices n and p independently of one another are a molar number and vary from 0 to 30, preferably from 1 to 25, and more preferably from 3 to 20, with the proviso that the sum n+p is greater than or equal to 1, preferably greater than 1, and less than or equal to 30, preferably less than 25, more preferably less than 20, and with particular preference less than 15, and

### B) one or more comonomers of the formula (2a), where R³² is hydrogen, Z is —C(CH₃)₂—CH₂—, and X is a hydrogen, ammonium, alkali metal or alkaline earth metal ion, in particular an ammonium or sodium ion,

and only in the case of the crosslinked polymers of component II)

### C) additionally trimethylolpropane triacrylate as crosslinker.

In one particularly preferred embodiment of the invention the preparations comprise one or more noncrosslinked copolymers of component I) and one or more crosslinked polymers of component II), in each case preparable by free-radical copolymerization of

### A) one or more macromonomers selected from esters of (meth)acrylic acid with

### (C₁₀-C₁₈)-fatty alcohol polyglycol ether with 8 EO units (Genapol® C-080)

### C₁₁ oxo-process alcohol polyglycol ether with 8 EO units (Genapol® UD-080)

### (C₁₂-C₁₄)-fatty alcohol polyglycol ether with 7 EO units (Genapol® LA-070)

### (C₁₂-C₁₄)-fatty alcohol polyglycol ether with 11 EO units (Genapol® LA-110)

### (C₁₆-C₁₈)-fatty alcohol polyglycol ether with 8 EO units (Genapol® T-080)

### (C16-C₁₈)-fatty alcohol polyglycol ether with 15 EO units (Genapol® T-150)

### (C₁₆-C₁₈)-fatty alcohol polyglycol ether with 11 EO units (Genapol® T-110)

### (C₁₆-C₁₈)-fatty alcohol polyglycol ether with 20 EO units (Genapol® T-200)

### (C₁₆-C₁₈)-fatty alcohol polyglycol ether with 25 EO units (Genapol® T-250)

### (C₁₈-C₂₂)-fatty alcohol polyglycol ether with 25 EO units and/or iso-(C₁₆-C₁₈)-fatty alcohol polyglycol ether with 25 EO units and

### B) one or more comonomers selected from acrylamidopropylmethylenesulfonic acid and/or its sodium or ammonium salt,

and only in the case of the crosslinked polymers of component II)

### C) additionally one or more crosslinkers, particularly trimethylolpropane triacrylate.

The Genapol® grades are products of the company Clariant.

In a further particularly preferred embodiment of the invention the preparations of the invention comprise one or more noncrosslinked copolymers of component I) and one or more crosslinked polymers of component II), in each case preparable by copolymerization of

### A) macromonomers of the formula (5) where p is 0 and n is a number from 7 to 25, R²⁷ is methyl, and R²⁹ is an alkyl group having 12 to 14 carbon atoms or 16 to 18 carbon atoms, and

### B) one or more comonomers selected from acrylamidopropylmethylenesulfonic acid and/or its salts with sodium ions or ammonium ions,

and only in the case of the crosslinked polymers of component II)

### C) additionally one or more crosslinkers, particularly trimethylolpropane triacrylate.

In a further preferred embodiment of the invention the one or more water-soluble noncrosslinked copolymers of component I) and/or the one or more water-soluble or water-swellable crosslinked or noncrosslinked copolymeric or homopolymeric thickeners of component II) contain one or more further structural units originating from one or more monomers selected from olefinically unsaturated acids and their salts with monovalent and divalent counterions, such as styrenesulfonic acid, vinylsulfonic acid, vinylphosphonic acid, allylsulfonic acid, methallylsulfonic acid, acrylic acid, (meth)acrylic acid, maleic acid and maleic anhydride and salts thereof; esters of (meth)acrylic acid with aliphatic, aromatic or cycloaliphatic alcohols having a carbon number from 1 to 22; esters of (meth)acrylic acid with alkyl ethoxylates, open-chain and cyclic N-vinyl amides (N-vinyllactams) having a ring size of 4 to 9 atoms, more preferably N-vinylformamide (NVF), N-vinylmethylformamide, N-vinylmethylacetamide (VIMA), N-vinylacetamide, N-vinylpyrrolidone (NVP), and N-vinylcaprolactam; amides of acrylic and of methacrylic acid, more preferably acrylamide, N,N-dimethylacrylamide, N,N-diethylacrylamide, alkoxylated acrylamides and methacrylamides, such as MAPTAC and APTAC; 2-vinylpyridine; 4-vinylpyridine; vinyl acetate; glycidyl methacrylate; acrylonitrile; vinyl chloride; vinylidene chloride; tetrafluoroethylene and/or DADMAC.

Suitable counterions for the salts of the olefinically unsaturated acids are preferably lithium, sodium, potassium, magnesium, calcium, ammonium, monoalkylammonium, dialkylammonium, trialkylammonium or tetraalkylammonium, the alkyl substituents of the ammonium ions being independently of one another (C₁-C₂₂)-alkyl radicals, which may be occupied by 0 to 3 hydroxyalkyl groups whose alkyl chain length may vary in a range from C₂ to C₁₀. Likewise suitable are singly to triply ethoxylated ammonium compounds with different degrees of ethoxylation. Particularly preferred counterions are sodium and ammonium. The degree of neutralization of the olefinically unsaturated acids is preferably 70 to 100 mol %.

The monomer distribution of the monomers A) and comonomers B) in the polymers of component I) and of component II), respectively, may for example be alternating, blockwise (including multiblock) or else statistical (including gradient).

The polymers present in the preparations of the invention have in general a number-average molecular weight from 1000 to 20 000 000, preferably from 20 000 to 5 000 000, and with particular preference from 100 000 to 1 500 000 g/mol.

The combination of noncrosslinked acryloyidimethyltaurine polymers of component I), preferably of the corresponding noncrosslinked hydrophobically modified polymers of component I), with crosslinked water-soluble or water-swellable copolymers of component II), based on acrylamidoalkylsulfonic acids and cyclic N-vinylcarboxamides and/or linear N-vinylcarboxamides, shows a thickening action in a synergistic way (see Table 1).

TABLE 1
Viscosities [mPa * s, 25° C., distilled H₂O (Brookfield, 20 rpm)] of individual
polymers and polymer combinations (all % figures in Table 1 are % by weight)

| | | | | | | Aqueous gel |
|---|---|---|---|---|---|---|
| | | | | | | with 0.5% |
| | | Aqueous | | Aqueous gel | Aqueous gel | Aristoflex ® |
| | Aqueous | gel with | Aqueous gel | with 0.5% | with 0.5% | AVC/0.5% |
| | gel with | 0.5% | with 0.5% | copolymer of | Aristoflex ® | copolymer of |
| | 0.5% | copolymer | Aristoflex ® | Example 3 | AVC/0.5% | Example 3 |
| Polymer | Aristoflex ® | of | AVC and | and 0.1% | copolymer of | and 0.1% |
| gel | AVC | Example 3 | 0.1% NaCl | NaCl | Example 3 | NaCl |
| Viscosity | 17017 | about 20 | 85 | 28 | 2900 | 5350 |
| (mPas) | | | | | | |

The values in Table 1 demonstrate a synergistic increase in the viscosities following electrolyte addition in the presence of the polymer combination, as compared with the polymers individually.

The polymers of component I) that are used in the cosmetic, pharmaceutical, and dermatological preparations of the invention are prepared by the processes described in EP 1 069 142.

In a further preferred embodiment of the invention the weight ratio of the noncrosslinked polymers of component I), preferably of the corresponding noncrosslinked hydrophobically modified polymers of component I), to the one or more water-soluble or water-swellable polymeric thickeners of component II) is in the range from 1 to 99:99 to 1, preferably in the range from 10 to 90:90 to 10, more preferably in the range from 20 to 80:80 to 20, and with particular preference in the range from 30 to 70:70 to 30.

The preparations of the invention contain the polymer mixture of components I) and II) preferably in an amount of from 0.1% to 10% by weight.

The viscosities of the 1% strength by weight aqueous solutions comprising at least one polymer of component I) and at least one polymer of component II) are preferably 500 to 50 000 mPas, in particular 1000 to 40 000 mPas, more preferably 2000 to 20 000 mPas at 25° C. (measured by Brookfield).

Even at room temperature, such polymers display a high thickener performance, effective emulsifying properties, and effective dispersion properties in aqueous, aqueous-alcoholic, and aqueous-surfactant solution or in emulsions.

Furthermore, preparations comprising such polymers display good transparency and high electrolyte stability.

The mixtures of water-soluble noncrosslinked polymers of component I) and water-soluble crosslinked polymers of component II) that are present in the preparations of the invention are suitable as thickeners and dispersants for aqueous preparations, aqueous-alcoholic and aqueous-surfactant preparations, and as emulsifiers, suspension agents with thickening effect, and consistency agents for emulsions and suspensions.

In a further preferred embodiment of the invention the preparations are in the form of a hair treatment, haircare, hairstyling or hair cleaning composition.

In a further preferred embodiment of the invention the preparations are in the form of an aqueous, gellike cosmetic or pharmaceutical composition.

Further preferred embodiments of the preparations of the invention are rinses, treatments, spray treatments, lotions, creams, styling creams for example, emulsions, gels, such as aqueous refreshing gels, mild cleansing gels, antiaging gels, and sunscreen gels, foams, mousses, fluids, and sprays, especially hair conditioners, shampoos, volume sprays, styling fluids, hair foams, hair gels, setting agents, hair sprays, mousses, hair oils, hair waxes, and split-end repair and prevention fluids.

In a further preferred embodiment of the invention the cosmetic and pharmaceutical preparations are surfactant-free compositions, surfactant-free emulsions, gels, sprays, spray foams, mousses or fluids.

In one particularly preferred embodiment of the invention the polymers of components I) and II) are incorporated into sprayable, pumpable, and foamable gels and foams, particularly into sprayable hair gels and foamable sun protection compositions, and bring about an improvement in the spraying characteristics of the compositions, with an optimized droplet-size distribution.

One advantageous composition is a hair gel composition comprising one or more polymers of component I) and one or more polymers of component II) and at least one hairsetting polymer.

The viscosity of the gels is preferably 100 to 5000 mPa*s, more preferably 200 to 1000 mPa*s, with particular preference 250 to 800 mPa*s, measured as a dynamic viscosity measurement using a Bohlin rheometer CS, measuring element C25, at a temperature of 25° C. and a shear rate of 50 s⁻¹.

The polymer mixture of the polymers of components I) and II) is used preferably in an amount of 0.1% to 10%, more preferably of 0.2% to 8% by weight, and the hairsetting polymer in an amount of preferably 0.1% to 15%, more preferably of 0.5% to 10% by weight.

The hairsetting polymer may be nonionic, anionic, cationic or amphoteric, but is preferably nonionic or anionic. It may be a synthetic or a natural polymer. Natural polymers are taken to include chemically modified polymers of natural origin. Preferred polymers in particular are those which possess sufficient solubility in water, alcohol or water/alcohol mixtures to be present in fully dissolved form in the composition of the invention. By hairsetting polymers are meant those polymers which on application to the hair as a 0.01% to 15% strength by weight aqueous, alcoholic or aqueous-alcoholic solution or dispersion are capable of producing a hairsetting effect.

Suitable synthetic, nonionic hairsetting polymers are homopolymers or copolymers constructed from at least one of the following monomers: vinylpyrrolidone, vinylimidazole, vinylcaprolactam, vinyl esters such as vinyl acetate, vinyl alcohol, acrylamide, methacrylamide, alkyl- and dialkylacrylamide, alkyl- and dialkylmethacrylamide, dialkylaminoalkylmethacrylamide, dialkylaminoalkylacrylamide, alkyl acrylate, alkyl methacrylate, propylene glycol or ethylene glycol, the alkyl groups of these monomers being C₁- to C₁₈-alkyl groups, preferably C₁- to C₇-alkyl groups, more preferably C₁- to C₃-alkyl groups. Suitable examples include homopolymers of vinylcaprolactam, of vinylpyrrolidone or of N-vinylformamide. Examples of further suitable hairsetting polymers are copolymers of vinylpyrrolidone and vinyl acetate, terpolymers of vinylpyrrolidone, vinyl acetate, and vinyl propionate, terpolymers of vinylpyrrolidone, vinylcaprolactam, and dialkylaminoalkyl(meth)acrylate, terpolymers of vinylpyrrolidone, vinylcaprolactam, and dialkylaminoalkyl(meth)acrylamide, terpolymers of vinylpyrrolidone, vinylimidazole, and (meth)acrylamide, polyacrylamide, polyvinyl alcohol, and also hairsetting polyethylene glycol/polypropylene glycol copolymers. Particularly preferred nonionic polymers are polyvinylpyrrolidone and polyvinylpyrrolidone/vinyl acetate copolymers. Preference is given to nonionic vinyllactam homopolymers and copolymers. Examples of suitable vinyllactams include vinylcaprolactam and vinylpyrrolidone. Particular preference is given to polyvinylpyrrolidone, polyvinylcaprolactam, terpolymers of vinylpyrrolidone, vinylimidazole, and (meth)acrylamide, and vinylpyrrolidone/vinyl acetate copolymers. Preferred commercial products are Luviskol® K 30, Luviskol® K 90, Luviskol® VA 37, Luviskol® VA 64, and Luvisete Clear.

Suitable anionic hairsetting polymers may be natural or synthetic homopolymers or copolymers with monomer units containing acid groups, copolymerized if appropriate with comonomers containing no acid groups. The acid groups are preferably selected from —COOH, —SO₃H, —OSO₃H, —OPO₂H, and —OPO₃H₂, among which the carboxylic acid groups are preferred. The acid groups may be in unneutralized form or in partly or fully neutralized form. They are preferably present from 50% to 100% in anionic or neutralized form. Neutralizing agents which can be used are those specified above. Suitable monomers are unsaturated, free-radically polymerizable compounds which carry at least one acid group, especially carboxyvinyl monomers. Suitable monomers containing acid groups are, for example, acrylic acid, methacrylic acid, crotonic acid, maleic acid or maleic anhydride or their monoesters, aldehydocarboxylic acids or ketocarboxylic acids.

Examples of comonomers not substituted by acid groups are acrylamide, methacrylamide, alkyl- and dialkylacrylamide, alkyl- and dialkylmethacrylamide, alkyl acrylate, alkyl methacrylate, vinylcaprolactone, vinylpyrrolidone, vinyl esters, vinyl alcohol, propylene glycol or ethylene glycol, amine-substituted vinyl monomers such as dialkylaminoalkyl acrylate, dialkylaminoalkyl methacrylate, monoalkylaminoalkyl acrylate, and monoalkylaminoalkyl methacrylate, the alkyl groups of these monomers being C₁- to C₁₈-alkyl groups, preferably C₁- to C₇-alkyl groups, more preferably C₁- to C₃-alkyl groups.

Suitable anionic polymers are, in particular, copolymers of acrylic acid or methacrylic acid with monomers selected from acrylic or methacrylic esters, acrylamides, methacrylamides, and vinylpyrrolidone, homopolymers of crotonic acid, and copolymers of crotonic acid with monomers selected from vinyl esters, acrylic or methacrylic esters, acrylamides, and methacrylamides. An example of a suitable natural polymer is shellac.

Preferred anionic polymers are crosslinked or noncrosslinked vinyl acetate/crotonic acid copolymers. Preference is likewise given to partially esterified copolymers of vinyl methyl ether with maleic anhydride. Further suitable anionic polymers are, for example, terpolymers of acrylic acid, alkyl acrylate, and N-alkylacrylamide, especially acrylic acid/ethyl acrylate/N-t-butylacrylamide terpolymers, or terpolymers of vinyl acetate, crotonate, and vinyl alkanoate, especially vinyl acetate/crotonate/vinyl neodecanoate copolymers.

Suitable film-forming amphoteric polymers are polymers which in addition to acidic or anionic groups contain, as further functional groups, basic or cationic groups, especially primary, secondary, tertiary or quaternary amine groups. Examples of these are copolymers formed from alkylacrylamide, alkylaminoalkyl methacrylate, and two or more monomers selected from acrylic acid, methacrylic acid or their esters, the alkyl groups containing 1 to 4 carbon atoms and at least one of the monomers containing an acid group.

Further examples of suitable hairsetting polymers are copolymers of acrylic acid, methacrylate, and methacrylamidopropyltrimethylammonium chloride, copolymers of acrylamidopropyltrimethylammonium chloride and acrylates, copolymers of acrylamide, acrylamidopropyltrimethylammonium chloride, 2-amidopropylacrylamide sulfonate, and dimethylaminopropylamine or chitosans. Also suitable are polymers with monomers which carry betaine groups, such as copolymers of methacryloylethylbetaine and two or more monomers of acrylic acid or its simple esters, known under the INCI name Methacryloyl Ethyl Betaine/Acrylate Copolymer.

In one preferred embodiment the preparation of the invention is formulated in an aqueous medium, in an alcoholic medium or in an aqueous-alcoholic medium containing preferably at least 10% by weight, more preferably at least 50% by weight, of water and, preferably, not more than 40% by weight of alcohol.

Alcohols present may in particular be the lower monoalcohols having 1 to 4 carbon atoms that are commonly used for cosmetic purposes, such as ethanol and isopropanol.

In a further preferred embodiment of the invention the preparations of the invention are in the form of a hair gel, and are preferably clear, transparent or translucent, colorless compositions.

In a further preferred embodiment of the invention the preparations are in sprayable form. These preparations have particularly positive spraying properties.

In one particularly preferred embodiment of the invention the preparations comprise one or more film formers. In this case they are preferably in the form of haircare compositions and cleansing compositions.

Preferred film formers, depending on the intended application, are salts of phenylbenzimidazolesulfonic acid, water-soluble polyurethanes, examples being C₁₀-polycarbamyl polyglyceryl esters, polyvinyl alcohol, polyvinylpyrrolidone copolymers, such as vinylpyrrolidone/vinyl acetate copolymer, water-soluble acrylic acid polymers/copolymers and their esters or salts, examples being partial ester copolymers of acrylic/methacrylic acid, and polyethylene glycol ethers of fatty alcohols, such as Acrylate/Steareth-20-Methacrylate Copolymer, water-soluble cellulose, such as hydroxymethylcellulose, hydroxyethylcellulose, and hydroxypropylcellulose, water-soluble quaterniums, polyquaterniums, carboxyvinyl polymers, such as carbomers and their salts, polysaccharides, such as polydextrose and glucan, vinyl acetate/crotonate, available for example under the trade name Aristoflex® A 60 (Clariant), and polymeric amine oxides, examples being representatives obtainable under the trade names Diaformer® Z-711, 712, 731, 651, 632, and 772 (Mitsubishi Chemical).

The hair treatment compositions of the invention contain preferably 0.01% to 15% by weight, more preferably 0.1% to 10% by weight, and with particular preference 1% to 5% by weight of film formers, based on the completed compositions.

In a further particularly preferred embodiment of the invention the cosmetic, pharmaceutical, and dermatological preparations comprise one or more UV filters.

Preferably, suitable UV filters include 4-aminobenzoic acid; 3-(4′-trimethylammonium)benzylideneboran-2-one methyl sulfate; 3,3,5-trimethylcyclohexyl salicylate; 2-hydroxy-4-methoxybenzophenone; 2-phenylbenzimidazole-5-sulfonic acid and its potassium, sodium, and triethanolamine salts; 3,3′-(1,4-phenylenedimethine)bis(7,7-dimethyl-2-oxobicyclo[2.2.1]heptane-1-methanesulfonic acid and its salts; 1-(4-tert-butylphenyl)-3-(4-methoxyphenyl)propane-1,3-dione, 3-(4′-sulfo)benzylidenebornan-2-one and its salts; 2-ethylhexyl 2-cyano-3,3-diphenylacrylate; polymer of N-[2(and 4)-(2-oxoborn-3-ylidenemethyl)benzyl]acrylamide; 2-ethylhexyl 4-methoxycinnamate; ethoxylated ethyl-4-aminobenzoate; isoamyl 4-methoxycinnamate; 2,4,6-tris[p-(2-ethylhexyloxycarbonyl)anilino]-1,3,5-triazine; 2-(2H-benzotriazol-2-yl)-4-methyl-6-(2-methyl-3-(1,3,3,3-tetramethyl-1-(trimethylsilyloxy)disiloxanyl)propyl)phenol; 4,4′-[(6-[4-((1,1-dimethylethyl)aminocarbonyl)phenylamino]-1,3,5-triazin-2,4-yl)diimino]bis(2-ethylhexyl benzoate); 3-(4′-methylbenzylidene)-D,L-camphor; 3-benzylidenecamphor; 2-ethylhexyl salicylate; 2-ethylhexyl 4-dimethylaminobenzoate; hydroxy-4-methoxybenzophenone-5-sulfonic acid (sulisobenzonum) and the sodium salt; and/or 4-isopropylbenzyl salicylate.

The cosmetic, pharmaceutical, and dermatological preparations of the invention contain UV filters preferably in the amounts of 0.0001% to 5% by weight, more preferably of 0.001% to 2% by weight, and with particular preference of 0.01% to 1% by weight, based on the completed preparations.

In a further preferred embodiment of the invention the cosmetic, pharmaceutical, and dermatological preparations comprise one or more antioxidants.

Advantageously the antioxidants are selected from the group consisting of amino acids (e.g., glycine, histidine, tyrosine, tryptophan) and their derivatives, imidazoles (e.g., urocaninic acid) and their derivatives, peptides such as D,L-carnosine, D-carnosine, L-carnosine and their derivatives (e.g., anserine), carotenoids, carotenes (e.g., α-carotene, β-carotene, lycopene) and their derivatives, chlorogenic acid and its derivatives, lipoic acid and its derivatives (e.g., dihydrolipoic acid), aurothioglucose, propylthiouracil and other thiols (e.g., thioredoxin, glutathione, cysteine, cystine, cystamine and their glycosyl, N-acetyl, methyl, ethyl, propyl, amyl, butyl and lauryl, palmitoyl, oleyl, γ-linoleyl, cholesteryl, and glyceryl esters) and also their salts, dilauryl thiodipropionate, distearyl thiodipropionate, thiodipropionic acid and derivatives thereof (esters, ethers, peptides, lipids, nucleotides, nucleosides, and salts) and also sulfoximine compounds (e.g., buthionine sulfoximines, homocysteine sulfoximine, buthionine sulfones, penta-, hexa-, and heptathionine sulfoximine) in very low tolerable doses (e.g., pmol/kg), and also (metal) chelators (e.g., α-hydroxy fatty acids, palmitic acid, phytic acid, lactoferrin), α-hydroxy acids (e.g., citric acid, lactic acid, malic acid), humic acid, bile acid, bile extracts, bilirubin, biliverdin, EDTA, EGTA and their derivatives, unsaturated fatty acids and their derivatives (e.g., γ-linolenic acid, linoleic acid, oleic acid), folic acid and its derivatives, ubiquinone and ubiquinol and their derivatives, vitamin C and derivatives (e.g., ascorbyl palmitate, Mg ascorbyl phosphate, ascorbyl acetate), tocopherols and derivatives (e.g., vitamin E acetate), vitamin A and derivatives (vitamin A palmitate), and also coniferyl benzoate of benzoin resin, rutic acid and its derivatives, α-glycosylrutin, ferulic acid, furfurylideneglucitol, carnosine, butylated hydroxytoluene, butylated hydroxyanisole, nordihydroguaiac resin acid, nordihydroguaiaretic acid, trihydroxybutyrophenone, uric acid and its derivatives, mannose and its derivatives, zinc and its derivatives (e.g., ZnO, ZnSO₄), selenium and its derivatives (e.g., selenomethionine), stilbenes and their derivatives (e.g., stilbene oxide, trans-stilbene oxide), superoxide dismutase, and the derivatives suitable in accordance with the invention (salts, esters, ethers, sugars, nucleotides, nucleosides, peptides, and lipids) of these stated substances.

With particular advantage for the purposes of the present invention it is possible to use water-soluble antioxidants.

The antioxidants can protect the hair and the skin against oxidative stress. Preferred antioxidants here are vitamin E and its derivatives and also vitamin A and its derivatives.

The amount of the antioxidants (one or more compounds) in the preparations of the invention is preferably 0.001% to 30% by weight, more preferably 0.05% to 20% by weight, and with particular preference 1% to 10% by weight, based on the total weight of the preparations.

Where vitamin E and/or its derivatives constitute the antioxidant or antioxidants, it is advantageous to select their respective concentrations from the range from 0.001% to 10% by weight, based on the total weight of the completed preparation.

Where vitamin A, or vitamin A derivatives, or carotenes or their derivatives constitute the antioxidant or antioxidants, it is advantageous to select their respective concentrations from the range from 0.001% to 10% by weight, based on the total weight of the preparation.

In one particularly preferred embodiment of the invention the cosmetic or pharmaceutical preparations comprise antioxidants selected from superoxide dismutase, tocopherol (vitamin E), and ascorbic acid (vitamin C).

In further embodiments of the invention the cosmetic, pharmaceutical, and dermatological preparations of the invention comprise active antimicrobials.

Of preferential suitability as active antimicrobials are cetyltrimethylammonium chloride, cetylpyridinium chloride, benzethonium chloride, diisobutylethoxyethyld imethylbenzylammonium chloride, sodium N-laurylsarcosinate, sodium N-palmethylsarcosinate, lauroylsarcosine, N-myristoylglycine, potassium N-laurylsarcosine, trimethylammonium chloride, sodium aluminum chlorohydroxylactate, triethyl citrate, tricetylmethylammonium chloride, 2,4,4′-trichloro-2′-hydroxydiphenyl ether (triclosan), phenoxyethanol, 1,5-pentanediol, 1,6-hexanediol, 3,4,4′-trichlorocarbanilide (triclocarban), diaminoalkylamide, such as L-lysine hexadecyl amide, citrate heavy metal salts, salicylates, piroctose, especially zinc salts, pyrithiones and their heavy metal salts, particularly zinc pyrithione, zinc phenol sulfate, farnesol, and combinations of these active substances.

The preparations of the invention contain the antimicrobial agents preferably in amounts up to 50% by weight, more preferably in amounts from 0.01% to 10% by weight, with particular preference in amounts from 0.1% to 10% by weight.

The water-soluble noncrosslinked and crosslinked polymers of components I) and II) that are used in the preparations of the invention are extremely compatible with pearlizing components. The hair treatment compositions of the invention may thus advantageously comprise pearlizing compounds, examples being fatty acid monoalkanolamides, fatty acid dialkanolamides, monoesters or diesters of alkylene glycol, especially ethylene glycol and/or propylene glycol or its oligomers with higher fatty acids, such as palmitic acid, stearic acid or behenic acid or mixtures thereof, monoesters or diesters of alkylene glycols with fatty acids, fatty acids and their metal salts, monoesters or polyesters of glycerol with carboxylic acids and keto sulfones of various kinds, preferably ethylene glycol distearate and polyethylene glycol distearate having approximately 3 glycol units.

The hair treatment compositions of the invention contain preferably 0.1% to 15%, more preferably 1% to 10% by weight of pearlizing compounds.

Glitter and luster effects in the preparations of the invention can be brought about preferably through addition of mica, colored polyacrylic esters and micas, micaceous iron oxide, micaceous titanium oxide, and by pigments. Suitable pigments include metal oxides, such as iron oxides, titanium oxide, ultramarine blue, and also pigments modified with cationic coating shells, as described in WO 00/12053 and EP 504 066.

The thickening and consistency-imparting effect of the polymers of components I) and II) that are used in the preparations of the invention is developed with particular advantage in the presence of nonionic and amphoteric surfactants.

Suitable nonionic surfactants, which can be used as detersive substances, include, preferably, fatty alcohol ethoxylates (alkylpolyethylene glycols); alkylphenolpolyethylene glycols; alkyl mercaptan polyethylene glycols; fatty amine ethoxylates (alkylaminopolyethylene glycols); fatty acid ethoxylates (acylpolyethylene glycols); polypropylene glycol ethoxylates (Pluronics®); fatty acid amide polyethylene glycols; N-alkoxypolyhydroxy fatty acid amide, especially fatty acid N-methylglucamides, sucrose esters; polyglycol ethers, alkylpolyglycosides, phosphoric esters (mono-, di-, and triphosphoric esters, ethoxylated and nonethoxylated).

The weight fraction of the nonionic surfactants in the preparations of the invention (in the case of rinse-off products, for example) is preferably in the range from 1% to 20% by weight, more preferably from 2% to 10% by weight, with particular preference from 3% to 7% by weight, based on the completed preparation.

Preferred amphoteric surfactants are: N—(C₁₂-C₁₈-alkyl)-β-aminopropionates and N—(C₁₂-C₁₈-alkyl)-β-iminodipropionates in the form of the alkali metal and mono-, di-, and trialkylammonium salts; N-acylaminoalkyl-N,N-dimethylacetobetaine, preferably N—(C₈-C₁₈-acyl)aminopropyl-N,N-dimethylacetobetaine; C₁₂-C₁₈-alkyldimethyl-sulfopropylbetaine; amphoteric surfactants based on imidazoline (trade name: Miranol®, Steinapon®), preferably the sodium salt of 1-(β-carboxymethyloxyethyl)-1-(carboxymethyl)-2-laurylimidazolinium; amine oxides, e.g., C₁₂-C₁₈-alkyldimethylamine oxide, fatty acid amidoalkyldimethylamine oxide, alkyltaurates, especially sodium methylcocoyltaurate (Hostapon CT, Clariant GmbH), sodium methyllauroyltaurate, and isethionates, such as sodium cocoylisethionate.

The weight fraction of the amphoteric surfactants is preferably 0.5% to 20% by weight, more preferably 1% to 10% by weight, based on the completed preparation.

In a further preferred embodiment the preparations of the invention are oil-in-water emulsions having a water fraction of 5% to 95%, preferably 15% to 75%, more preferably 25% to 85% by weight.

Preparations of the invention that are in emulsion form may comprise one or more emulsifiers. These emulsifiers may be selected from the group of nonionic, anionic, cationic or amphoteric emulsifiers.

Suitable nonionic emulsifiers are adducts of 2 to 30 mol of ethylene oxide and/or up to 5 mol of propylene oxide with linear fatty alcohols having 8 to 22 carbon atoms, with fatty acids having 12 to 22 carbon atoms, and with alkylphenols having 8 to 15 carbon atoms in the alkyl group; C₁₂-C₁₈-fatty acid monoesters and diesters of adducts of 1 to 30 mol of ethylene oxide with glycerol; glyceryl monoesters and diesters and sorbitol monoesters and diesters of saturated and unsaturated fatty acids having 6 to 22 carbon atoms and their ethylene oxide adducts; adducts of 15 to 60 mol of ethylene oxide with castor oil and/or hydrogenated castor oil; polyol esters and especially polyglyceryl esters such as polyglyceryl polyricinoleate and polyglyceryl poly-12-hydroxystearate, for example. Preferred liquid fatty acid esters are PEG-10 Polyglyceryl-2 Laurate and Polyglyceryl-2 Sesquiisostearate.

Examples of suitable ionogenic emulsifiers include anionic emulsifiers, such as mono-, di- or tri-phosphoric esters, soaps (e.g., sodium stearate), and fatty alcohol sulfates, but especially cationic emulsifiers such as mono-, di-, and tri-alkyl quats and their polymeric derivatives.

Amphoteric emulsifiers available are preferably alkylaminoalkylcarboxylic acids, betaines, sulfobetaines, and imidazoline derivatives.

In addition it is possible to use naturally occurring emulsifiers, among which beeswax, lanolin, lecithin, and sterols are preferred.

Fatty alcohol ethoxylates are preferably selected from the group of ethoxylated stearyl alcohols, cetyl alcohols, cetylstearyl alcohols, especially polyethylene glycol(13) stearyl ether, polyethylene glycol(14) stearyl ether, polyethylene glycol(15) stearyl ether, polyethylene glycol(16) stearyl ether, polyethylene glycol(17) stearyl ether, polyethylene glycol(18) stearyl ether, polyethylene glycol(19) stearyl ether, polyethylene glycol(20) stearyl ether, polyethylene glycol(12) isostearyl ether, polyethylene glycol(13) isostearyl ether, polyethylene glycol(14) isostearyl ether, polyethylene glycol(15) isostearyl ether, polyethylene glycol(16) isostearyl ether, polyethylene glycol(17) isostearyl ether, polyethylene glycol(18) isostearyl ether, polyethylene glycol(19) isostearyl ether, polyethylene glycol(20) isostearyl ether, polyethylene glycol(13) cetyl ether, polyethylene glycol(14) cetyl ether, polyethylene glycol(15) cetyl ether, polyethylene glycol(16) cetyl ether, polyethylene glycol(17) cetyl ether, polyethylene glycol(18) cetyl ether, polyethylene glycol(19) cetyl ether, polyethylene glycol(20) cetyl ether, polyethylene glycol(13) isocetyl ether, polyethylene glycol(14) isocetyl ether, polyethylene glycol(15) isocetyl ether, polyethylene glycol(16) isocetyl ether, polyethylene glycol(17) isocetyl ether, polyethylene glycol(18) isocetyl ether, polyethylene glycol(19) isocetyl ether, polyethylene glycol(20) isocetyl ether, polyethylene glycol(12) oleyl ether, polyethylene glycol(13) oleyl ether, polyethylene glycol(14) oleyl ether, polyethylene glycol(15) oleyl ether, polyethylene glycol(12) lauryl ether, polyethylene glycol(12) isolauryl ether, polyethylene glycol(13) cetylstearyl ether, polyethylene glycol(14) cetylstearyl ether, polyethylene glycol(15) cetylstearyl ether, polyethylene glycol(16) cetylstearyl ether, polyethylene glycol(17) cetylstearyl ether, polyethylene glycol(18) cetylstearyl ether, polyethylene glycol(19) cetylstearyl ether, polyethylene glycol(20) cetylstearyl ether, polyethylene glycol(20) stearate, polyethylene glycol(21) stearate, polyethylene glycol(22) stearate, polyethylene glycol(23) stearate, polyethylene glycol(24) stearate, polyethylene glycol(25) stearate, polyethylene glycol(12) isostearate, polyethylene glycol(13) isostearate, polyethylene glycol(14) isostearate, polyethylene glycol(15) isostearate, polyethylene glycol(16) isostearate, polyethylene glycol(17) isostearate, polyethylene glycol(18) isostearate, polyethylene glycol(19) isostearate, polyethylene glycol(20) isostearate, polyethylene glycol(21) isostearate, polyethylene glycol(22) isostearate, polyethylene glycol(23) isostearate, polyethylene glycol(24) isostearate, polyethylene glycol(25) isostearate, polyethylene glycol(12) oleate, polyethylene glycol(13) oleate, polyethylene glycol(14) oleate, polyethylene glycol(15) oleate, polyethylene glycol(16) oleate, polyethylene glycol(17) oleate, polyethylene glycol(18) oleate, polyethylene glycol(19) oleate, polyethylene glycol(20) oleate.

In addition it is of advantage to select the polyethyl glycol glyceryl fatty acid esters from the group polyethylene glycol(20) glyceryl laurate, polyethylene glycol(6) glyceryl caprate/caprinate, polyethyl glycol(20) glyceryl oleate, polyethylene glycol(20) glyceryl isostearate, and polyethylene glycol(18) glyceryl oleate/cocoate.

Particularly suitable among the sorbitol esters are polyethylene glycol(20) sorbitol monolaurate, polyethylene glycol(20) sorbitol monostearate, polyethylene glycol(20) sorbitol monoisostearate, polyethylene glycol(20) sorbitol monopalmitate, and polyethylene glycol(20) sorbitol monooleate.

The weight fraction of the emulsifier or emulsifiers present in the preparations of the invention is preferably 0.1% to 20% by weight, more preferably 0.5% to 15% by weight, with particular preference 1% to 10% by weight, based on the completed preparation.

The weight fraction of the polymer mixture employed that comprises one or more polymers of component I) and one or more polymers of component II) in the emulsions is preferably 0.05% to 10% by weight, more preferably 0.1% to 5% by weight, and with particular preference 0.2% to 3% by weight, based on the completed preparations.

For the preparations of the invention on an aqueous-alcoholic or alcoholic basis, all monohydric or polyhydric alcohols are suitable. Preference is given to alcohols having 1 to 4 carbon atoms such as ethanol, propanol, isopropanol, n-butanol, isobutanol, tert-butanol or glycerol, and alkylene glycols, especially propylene, butylene or hexylene glycol, and mixtures of said alcohols. Further preferred alcohols are polyethylene glycols having a relative molecular mass below 2000. Particular preference is given to using polyethylene glycol having a relative molecular mass between 200 and 600 and polyethylene glycol having a relative molecular mass between 400 and 600.

The weight fraction of the polymer mixture comprising one or more polymers of component I) and one or more polymers of component II) in aqueous preparations or in preparations on an aqueous-alcoholic basis is 0.05% to 10% by weight, preferably 0.1% to 5% by weight, more preferably 0.2% to 3% by weight, based on the completed preparations.

Further preferred embodiments are aqueous-surfactant-based preparations.

Preference is given to anionic, nonionic, and amphoteric surfactants. As anionic detersive substances mention may be made with preference of the following: C₁₀-C₂₀ alkyl and alkylene carboxylates, alkyl ether carboxylates, fatty alcohol sulfates, fatty alcohol ether sulfates, alkylamidesulfates and alkylamidesulfonates, fatty acid alkylamide polyglycol ether sulfates, alkane sulfate, alkanesulfonates and hydroxyalkanesulfonates, olefinsulfonates, acyl esters of isethionates, α-sulfo fatty acid esters, alkylbenzenesulfonates, alkylphenol glycol ether sulfonates, sulfosuccinates, sulfosuccinic monoesters and diesters, fatty alcohol ether phosphates, protein-fatty acid condensates, alkylmonoglyceride sulfates and alkylmonoglyceridesulfonates, alkylglyceride ether sulfonates, fatty acid methyltaurides, fatty acid sarcosinates, sulforicinoleates, amphoacetates or amphoglycinates, acylglutamates, and anionically modified alkylpolyglucosides. These compounds and their mixtures are utilized in the form of their water-soluble or water-dispersible salts, examples being the sodium, potassium, magnesium, ammonium, mono-, di-, and triethanolammonium salts and also analogous alkylammonium salts.

The weight fraction of the anionic surfactants is preferably 1% to 30%, more preferably 5% to 25%, with particular preference 10% to 22%, by weight, based on the completed preparations.

Examples of suitable nonionic surfactants, which can be used as detersive substances, include the following: fatty alcohol ethoxylates (alkylpolyethylene glycols); alkylphenolpolyethylene glycols; alkyl mercaptan polyethylene glycols; fatty amine ethoxylates (alkylaminopolyethylene glycols); fatty acid ethoxylates (acylpolyethylene glycols); polypropylene glycol ethoxylates (Pluronics®); fatty acid alkylolamides, (fatty acid amide polyethylene glycols); N-alkyl-, N-alkoxypolyhydroxy fatty acid amide, sucrose esters; alkylpolyglucosides (APG®); sorbitol esters and the polyglycol ether.

The weight fraction of the nonionic surfactants in the preparations of the invention is preferably in the range from 1% to 20%, more preferably 2% to 10%, with particular preference 3% to 7% by weight.

Preferred amphoteric surfactants are: N—(C₁₂-C₁₈)-alkyl-β-aminopropionates and N—(C₁₂-C₁₈)-alkyl-β-iminodipropionates in the form of the alkali metal and mono-, di-, and trialkylammonium salts; N-acylaminoalkyl-N,N-dimethylacetobetaine, preferably N—(C₈-C₁₈)-acylaminopropyl-N,N-dimethylacetobetaine; (C₁₂-C₁₈)-alkyldimethyl-sulfopropylbetaine; amphoteric surfactants based on imidazoline (trade name: Miranol®, Steinapon®), preferably the sodium salt of 1-(β-carboxymethyloxyethyl)-1-(carboxymethyl)-2-laurylimidazolinium; amine oxide, e.g., (C₁₂-C₁₈)-alkyl-dimethylamine oxide, and fatty acid amidoalkyldimethylamine oxide.

The weight fraction of the amphoteric surfactants in the preparations of the invention is preferably in the range from 0.5% to 20% by weight, more preferably from 1% to 10% by weight. In the preparations of the invention it is possible in addition to use foam-boosting cosurfactants from the group of alkylbetaines, alkylamidobetaines, aminopropionates, aminoglycinates, imidazolinium betaines, and sulfobetaines, amine oxides and fatty acid alkanol amides or polyhydroxyamides.

Preferred surfactants in the preparations of the invention are alkylbetaines, especially cocoamidopropylbetaine, amphoacetates, acylglutamates, especially sodium cocoylglutamate, alkyl ether sulfosuccinates, especially disodium laureth-sulfosuccinate, cocoyidiethanolamide, sodium cocoylisethionate, sodium methylcocoyltaurate and sodium methyllauroyltaurate.

The total amount of surfactants used in the preparations of the invention is preferably 1% to 70%, more preferably 5% to 40%, with particular preference 12% to 35%, by weight, based on the completed preparation.

The weight fraction of the polymer mixture employed, comprising one or more polymers of component I) and one or more polymers of component II), in aqueous-surfactant preparations is 0.05% to 10%, preferably 0.1% to 5%, more preferably 0.2% to 3%, by weight, based on the completed preparations.

The cosmetic, pharmaceutical, and dermatological preparations of the invention may comprise, as further auxiliaries and additives, gelling agents, superfatting agents, moisturizers, silicones, stabilizers, conditioners, glycerol, preservatives, dyes, fragrance and perfume oils, solvents, hydrotropes, opacifiers, fatty alcohols, antidandruff agents, vitamins, bisabolol, allantoin, phytantriol, panthenol, AHA acids, plant extracts, aloe vera for example, self-tanning agents, such as dihydroxyacetone or erythrulose, and proteins.

The desired viscosity of the preparations can be set by adding further thickeners. Those suitable include, preferably, cellulose ethers and other cellulose derivatives (e.g., carboxymethylcellulose, hydroxyethylcellulose), gelatin, starch and starch derivatives, sodium alginates, fatty acid polyethylene glycol esters, agar agar, xanthan, guar and guar derivatives, scleroglucan, tragacanth or dextrin derivatives, especially dextrin esters.

Synthetic polymers employed include a variety of materials, preferably polyvinyl alcohols, polyacrylamides, polyvinylamides, polysulfonic acids, especially copolymers based on ammonium salts of acrylamidoalkylsulfonic acids and cyclic N-vinylcarboxamides and/or cyclic and linear N-vinylcarboxamides or else hydrophobically modified acrylamidoalkylsulfonic acid copolymers, polyacrylic acid, polyacrylic acid derivatives, polyacrylic esters, polyvinylpyrrolidone, polyvinyl methyl ether, polyethylene oxides, copolymers of maleic anhydride and vinyl methyl ether, and also various mixtures and copolymers of the abovementioned compounds, including their various salts and esters. These polymers may be alternatively crosslinked or noncrosslinked.

Suitable gelling agents include all surface-active substances which, in solution in the liquid phase, form a network structure and so solidify the liquid phase.

Suitable gelling agents are specified for example in WO 98/58625.

Preferred gelling agents are metal salts of fatty acids, preferably having 12 to 22 carbon atoms, examples being sodium stearate, sodium palmitate, sodium laurate, sodium arachidates, sodium behenate, potassium stearate, potassium palmitate, sodium myristate, aluminum monostearate, hydroxy fatty acids, examples being 12-hydroxystearic acid, 16-hydroxyhexadecanoyl acid; fatty acid amides; fatty acid alkanol amides; dibenzalsorbitol, and alcohol-soluble polyamides and polyacrylamides or mixtures of such.

Preferably the preparations of the invention contain 0.01% to 20%, more preferably 0.1% to 10%, with particular preference 1% to 8%, and with very particular preference 3% to 7% by weight of gelling agents.

Further additives may be silicone compounds, preferably dimethylpolysiloxanes, methylphenylpolysiloxanes, cyclic silicones, and also amino-, fatty acid-, alcohol-, polyether-, epoxy-, fluoro- and/or alkyl-modified silicone compounds, examples being phenyltrimethicones from Clariant GmbH such as SilCare® 15M30, SilCare® 15M40, SilCare® 15M50, SilCare® 15M60, caprylyltrimethicones such as SilCare® 31 M30, SilCare® 31 M40, SilCare® 31 M 50, SilCare® 31 M 60, alkylmethicones such as SilCare® Silicone 41 M10, SilCare® Silicone 41 M15, SilCare® Silicone 41 M20, SilCare® Silicone 41 M30, SilCare® 41 M40, SilCare® 41 M50, SilCare® 41 M65, SilCare® 41 M70 or SilCare® 41 M80, SilCare® 41 M90, trimethylsilyl trimethylsiloxylactate, trimethylsilyl trimethylsiloxyglycolate, trimethylsilyl trimethylsiloxysalicylate, retinoxytrimethylsilane, polyalkylarylsiloxanes and polyethersiloxane copolymers, and modified polyorganosiloxanes such as SilCare® Silicone SEA (Clariant GmbH).

The preparations of the invention may contain the abovementioned silicone compounds preferably in the amounts by weight of 0.1% to 20%, more preferably of 0.2% to 15%, and with particular preference 0.5% to 10% by weight, based on the completed preparations.

Suitable carrier materials include preferably vegetable oils, natural and hydrogenated oils, waxes, fats, water, alcohols, polyols, glycerol, glycerides, liquid paraffins, liquid fatty alcohols, sterol, polyethylene glycols, and cellulose and cellulose derivatives.

Fungicidal actives that can be used include preferably ketoconazole, oxiconazole, bifonazoles, butoconazoles, cloconazoles, clotrimazoles, econazoles, enilconazoles, fenticonazoles, isoconazoles, miconazoles, sulconazoles, tioconazoles, fluconazoles, itraconazoles, terconazoles, naftifins and terbinafins, Zn pyrithione and Octopiroxe in the amounts by weight of 0.05% to 5%, preferably 0.1% to 3%, more preferably 0.2% to 2%, by weight, based on the completed preparations.

The preparations of the invention may comprise organic acids, preferably α- or β-hydroxy acids, more preferably α-hydroxy acids.

Particular preference is given to α-hydroxy acids of the formula (6)

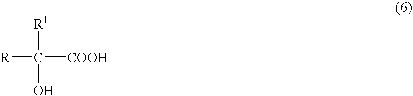

where R and R¹ independently of one another are H, F, Cl, Br, alkyl, aralkyl or aryl groups having saturated or unsaturated, linear or branched chains, cyclic groups or OH, CHO, COOH or alkoxy groups having 1 to 9 carbon atoms. It is possible to employ the acid and/or the corresponding salt with alkali metal or ammonium counterions.

Suitable acidic components include glycolic acid, lactic acid, methyllactone acid, 2-hydroxybutanoic, -pentanoic, -hexanoic, -heptanoic, -octanoic, -nonanoic, -decanoic, -undecanoic, and -dodecanoic acid (laurylic acid), α-hydroxymyristic acid, α-hydroxypalmitic acid, α-hydroxystearic acid, arachidonic acid, 2-phenyl-2-hydroxyethanoic acid, citric acid, tartaric acid, mandelic acid, salicylic acid, ascorbic acid, pyruvic acid, 2,2-diphenyl-2-hydroxyethanoic acid, 3-phenyl-2-hydroxypropanoic acid, 2-phenyl-2-methyl-2-hydroxyethanoic acid, 2-(4-hydroxyphenyl)-2-hydroxyethanoic acid, 2-(4′-d ichlorophenyl)-2-hydroxyethanoic acid, 2-(3′-hydroxy-4′-methoxyphenyl)-2-hydroxyethanoic acid, 2-(4′-hydroxy-3′-methoxyphenyl)-2-hydroxyethanoic acid, 3-(2′-hydroxyphenyl)-2-hydroxypropanoic acid, 3-(4′-hydroxyphenyl)-2-hydroxypropanoic acid, 2-(3′,4′-dihydroxyphenyl)-2-hydroxyethanoic acid, fumaric acid, retinoic acid, aliphatic and organic sulfonic acids, benzoic acid, kojic acid, fruit acid, malic acid, gluconic acid, galacturonic acid, acidic plant extracts and/or fruit extracts, and derivatives thereof.

In a further preferred embodiment the preparations contain, based on the total weight of the preparations, 0.01% to 20% by weight, preferably 0.5% to 10% by weight and more preferably 1% to 5% by weight of hydroxy acids, preferably α-hydroxy acids, which may also be present partly in salt form. In one particularly preferred embodiment these preparations contain, based on the total weight of the preparations, 0.01% to 10% by weight of polymer mixture comprising one or more polymers of component I) and one or more polymers of component II).

With particular preference the preparations of the invention comprise glycolic acid, lactic acid and/or 2-hydroxyoctanoic acid.

The hydroxy acid and the corresponding salt are preferably in a molar ratio in the range from 1000:1 to 1:1000, more preferably 50:1 to 1:50.

The preparations of the invention can be blended advantageously with conventional ceramides, pseudoceramides, fatty acid N-alkylpolyhydroxyalkyl amides, cholesterol, cholesterol fatty acid esters, fatty acids, triglycerides, cerebrosides, phospholipids, substances having a keratolytic and keratoplastic action, and similar substances.

As a moisturizing substance, preferably isopropyl palmitate, glycerol and/or sorbitol are available, and are employed preferably in the amounts by weight of 0.1% to 50%.

As self-tanning agents it is possible with preference to use dihydroxyacetone (DHA) and erythrulose in the amounts by weight of 0.1% to 10%, preferably 0.2% to 8%.

Superfatting agents which can be used are preferably lanolin and lecithin, unethoxylated and polyethoxylated or acylated lanolin derivatives and lecithin derivatives, polyol fatty acid esters, mono-, di-, and triglycerides and/or fatty acid alkanol amides.

Suitable preservatives include, preferably, phenoxyethanol, parabens, especially butylparaben, isobutylparabens, ethylparaben, methylparaben, propylparaben, pentanediol, imidazolidinylurea or sorbic acid.

Preference is given to employing the following commercial products:

Nipabutyl (Butylparaben), Nipagin (Ethylparaben), Nipagin M (Methylparaben), Nipasol M (Propylparaben), Nipabutyl sodium salt (Butylparaben, sodium salt), Nipagin A sodium salt (Ethylparaben, sodium salt), Nipagin M sodium salt (Methylparaben, sodium salt), Nipa Biopure 100 (Imidazolidinyl Urea), Nipa Biopure 200 (Diazolidinyl Urea), Nipaguard BNPD (2-Bromo-2-Nitropropane-1,3-diol), Nipaguard BNPD (2-Bromo-2-Nitropropane-1,3-diol), Nipaguard DMDMH (DMDM Hydantoin), Nipaguard SMG (Hydroxymethylglycinate, sodium salt), Phenoxetol (Phenoxyethanol), Propylene Phenoxetol (Phenoxyisopropanol), Nipasept (Methylparaben, Ethylparaben, Propylparaben), Nipastat (Methylparaben, Butylparaben, Ethylparaben, Propylparaben, Isobutylparaben), Nipasept Sodium (Methylparaben, sodium salt, Ethylparaben, sodium salt, Propylparaben, sodium salt), Nipastat Sodium (Butylparaben, sodium salt), Nipacide A (Methylparaben, Butylparaben, Isobutylparaben), Nipacide A Sodium (Methylparaben, sodium salt, Butylparaben, sodium salt, Isobutylparaben, sodium salt), Nipacombin A (Sodium Propylparaben, Sodium Methylparaben, Sodium Ethylparaben, Sodium Benzoates), Nipacombin SK (Sodium Propylparaben, Sodium Butylparaben), Nipaguard BPX (Phenoxyethanol, Methylparaben, Propylparaben, 2-Bromo-2-Nitropropane-1,3-diol), Nipaguard CMB (Triethylene Glycol, Benzyl Alcohol, Propylene Glycol, Methylchloroisothiazolinone and Methylisothiazolinone 3:1), Nipaguard DCB (Phenoxyethanol, Methyldibromo Glutaronitrile), Nipaguard IPF (PEG-4 Laurate, Iodopropynyl Butylcarbamate), Nipaguard IPP2 (Phenoxyethanol, Iodopropynyl Butylcarbamate), Nipaguard MPA (Benzyl Alcohol, Methylparaben, Propylparaben), Nipaguard MPS (Methylparaben, Propylparaben, Propylene Glycol), Nipaguard PBI (Iodopropynyl Butylcarbamate, Phenoxyethanol, Bronopol), Nipaguard PDU (Propylene Glycol, Diazolidinyl Urea, Methylparaben, Propylparaben), Nipaguard TBK (Phenoxyethanol, Methyldibromo Glutaronitrile, 2-Bromo-2-Nitropropane-1,3-diol, Butylparaben, Isobutylparaben), JM ActiCare (Silver Chloride, Titanium Dioxide, Diethylhexyl Sulfosuccinate, sodium salt, Propylene Glycol), Phenonip (Phenoxyethanol, Methylparaben, Ethylparaben, Butylparaben, Propylparaben, Isobutylparaben), Phenosept (Chloroxylenol, Phenoxyisopropanol), Phenosept PG (Chloroxylenol, Phenoxyisopropanol, Propylene Glycol), Nipaguard DCB (Phenoxyethanol, Methyldibromo Glutaronitrile), Nipaguard IPF (PEG-4 Laurate, Iodopropynyl Butylcarbamate), Nipaguard IPP2 (Phenoxyethanol, Iodopropynyl Butylcarbamate), Nipaguard MPA (Benzyl Alcohol, Methylparaben, Propylparaben), Nipaguard MPS (Methylparaben, Propylparaben, Propylene Glycol), Nipaguard PBI (Iodopropynyl Butylcarbamate, Phenoxyethanol, Bronopol), Nipaguard PDU (Propylene Glycol, Diazolidinyl Urea, Methylparaben, Propylparaben), Nipaguard TBK (Phenoxyethanol, Methyldibromo Glutaronitrile, 2-Bromo-2-Nitropropane-1,3-diol, Butylparaben, Isobutylparaben), JM ActiCare (Silver Chloride, Titanium Dioxide, Diethylhexyl Sodium Sulfosuccinate, Propylene Glycol), Phenonip (Phenoxyethanol, Methylparaben, Ethylparaben, Butylparaben, Propylparaben, Isobutylparaben), Phenosept (Chloroxylenol, Phenoxyisopropanol), Phenosept PG (Chloroxylenol, Phenoxyisopropanol, Propylene Glycol).

They are used preferably in the amounts by weight of 0.001% to 5%, more preferably of 0.01% to 3%, with particular preference of 0.1% to 2%, by weight, based on the completed preparations.

As dyes it is possible to use the substances that are approved and suitable for cosmetic and pharmaceutical use.

As fragrance or perfume oils it is possible to use individual odorant compounds, examples being the synthetic products of the ester, ether, aldehyde, ketone, alcohol, and hydrocarbon types. Odorant compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethyl methylphenylglycinate, allyl cyclohexylpropionate, styrallyl propionate and benzyl salicylate. The ethers include, for example, benzyl ethyl ether; the aldehydes include, for example, the linear alkanals having 8 to 18 carbon atoms, citral, citronellal, citronellyloxyacetaldehyde, cyclamenaldehyde, hydroxycitronellal, lilial and bourgeonal; the ketones include, for example, the ionones, alpha-isomethyl ionone and methyl cedryl ketone; the alcohols include anethole, citronellol, eugenol, geraniol, linalool, phenylethyl alcohol, and terpineol; the hydrocarbons include primarily the terpenes and balsams. It is preferred to use mixtures of different odorants which together produce a pleasing fragrance note.

Perfume oils may also comprise natural odorant mixtures, of the kind obtainable from plant or animal sources, examples being pine oil, citrus oil, jasmine oil, lily oil, rose oil or ylang-ylang oil. Essential oils of relatively low volatility, which are mostly used as aroma components, are also suitable as perfume oils, examples being sage oil, camomile oil, oil of cloves, melissa oil, mint oil, cinnamon leaf oil, lime blossom oil, juniper berry oil, vetiver oil, olibanum oil, galbanum oil, and labdanum oil.

As acids or alkalis for pH adjustment it is preferred to use mineral acids, HCl for example, inorganic bases, such as NaOH and KOH, and organic acids, preferably citric acid.

The preparations are preferably adjusted to a pH in the range 2 to 10, preferably pH 3 to 8, more preferably 4 to 7.

In a further preferred embodiment of the invention the preparations are clearly transparent or translucent.

In a further preferred embodiment of the invention the preparations are emulsifier-free and/or oil-free.

In a further preferred embodiment of the invention the preparations are nonsticky.

In a further preferred embodiment of the invention the preparations are gels and have a long-lasting gel texture on the skin or hair.

In a further preferred embodiment of the invention the preparations impart extensive sensorial properties.

The preparations of the invention impart a pleasant feeling on skin and hair. They are also notable for improved stability, particularly to electrolytes. Furthermore, they also have a very esthetic appearance.

In a further preferred embodiment of the invention the preparations comprise as component II) one or more water-soluble or water-swellable crosslinked copolymeric thickeners containing one or more structural units of the formula (1), one or more structural units of the formula (2), and one or more crosslinking structural units originating from monomers having at least two olefinic double bonds.

The applications and examples below are intended to illustrate the invention, but without restricting it to them (all percentages are by weight).

### EXAMPLES AND APPLICATIONS

### 1) Preparation of Macromonomers

### Version 1: Glycidyl Methacrylate

A one-liter three-neck flask with stirrer, internal thermometer, and reflux condenser is charged with 600 g of Genapol® T-250 and 75 g of glycidyl methacrylate are added. Subsequently the reaction mixture is heated at 100° C. for 2 hours and the excess glycidyl methacrylate is distilled off under reduced pressure. The resulting macromonomer can be used without further purification for the polymerization.

### Version 2: Free Meth-/Acrylic Acid

A one-liter three-neck flask with stirrer, internal thermometer, and reflux condenser is charged with 500 g of Genapol® UD-070, and 100 g of meth-/acrylic acid and p-toluenesulfonic acid catalyst are added. Subsequently the reaction mixture is boiled under reflux for 2 hours and the excess acid and the water of reaction formed are distilled off under reduced pressure. The resulting macromonomer can be used without further purification for the polymerization.

### Version 3: Halogen Derivatives of Meth-/Acrylic Acid

A one-liter three-neck flask with stirrer, internal thermometer, and reflux condenser is charged with 500 g of Genapol® UD-80 containing a primary amino end group, and 110 g of meth-/acryloyl chloride and 50 g of sodium carbonate are added. Subsequently the reaction mixture is boiled under reflux for 2 hours. The cessation of CO₂ evolution indicates the end of the modification reaction. The excess acid chloride is distilled off under reduced pressure. The resulting macromonomer with a meth-/acrylamide end group can be used without further purification for the polymerization.

### Version 4: Ester of Meth-/Acrylic Acid

A one-liter three-neck flask with stirrer, internal thermometer, and reflux condenser is charged with 500 g of Genapol® LA-070, and 100 g of methyl meth-/acrylate and 20 g of titanium tetraisopropoxide are added. Subsequently the reaction mixture is boiled under reflux for 2 hours. When the resulting alcohol has been removed by distillation, the remaining ester is distilled off under reduced pressure. The resulting macromonomer with a meth-/acrylic acid end group can be used without further purification for the polymerization.

### 2) Polymerization

General polymerization procedure for preparing the side chain polymers, used in the preparations of the invention, by the precipitation process in tert-butanol.

A 2-liter Quickfit flask with reflux condenser, gas inlet, internal thermometer, and stirrer is charged with 500 ml of tert-butanol, and the calculated amount of acryloyldimethyltaurine is added. Subsequently, neutralization is effected by introduction of NH₃, and the LCST (lower critical solution temperature) side arms prepared as under 1), i.e., the corresponding macromonomers (two or more different species also possible), are added to the reaction mixture. If further comonomers are needed, they can be added to the reaction mixture following neutralization. After the mixture has been rendered inert using N₂ or argon, AIBN (azobisisobutyronitrile) initiator is added at an internal temperature of 60° C. and the polymerization reaction is initiated. After a few minutes, the completed polymer is precipitated. The mixture is heated at reflux for two hours and the polymer is subsequently freed from the solvent on a suction filter and dried under reduced pressure. This procedure can be employed generally for all the polymerization reactions described below.

Noncrosslinked side chain polymers (polymers 1)

### Example 1

### Reaction as Per General Polymerization Procedure

| Reactant | Amount (g) |
|---|---|
| Macromonomer | 20 |
| of version 1 - type Genapol ® T-250 | |
| NH₃-neutralized acryloyldimethyltaurine | 100 |
| tert-Butanol | 300 |
| AIBN (initiator) | 1 |

### Example 2

### Reaction as Per General Polymerization Procedure

| Reactant | Amount (g) |
|---|---|
| Macromonomer | 15 |
| of version 3 - type Genapol ® UD-80 | |
| Macromonomer | 15 |
| of version 1 - type Genapol ® T-250 | |
| NH₃-neutralized acryloyldimethyitaurine | 90 |
| tert-Butanol | 300 |
| AIBN (initiator) | 1 |

### Example 3

### Reaction as Per General Polymerization Procedure

| Reactant | Amount (g) |
|---|---|
| Macromonomer | 18 |
| of version 4 - type Genapol ® LA-070 | |
| NH₃-neutral ized acryloyldimethyltaurine | 80 |
| tert-Butanol | 300 |
| Dilauroyl peroxide (DLP) (initiator) | 2 |

### Example 4

### Reaction as Per General Polymerization Procedure

The macromonomer from Example 4 is prepared in analogy to version 1 with the difference that, instead of 600 g of Genapol® T-250, 600 g of Genapol® LA-070 are used.

| Reactant | Amount (g) |
|---|---|
| Macromonomer | 20 |
| of version 1 - type Genapol ® LA-070 | |
| Na-neutralized acryloyldimethyltaurine | 75 |
| Acrylamide | 50 |
| tert-Butanol | 300 |
| AIBN (initiator) | 1 |

### Example 5

### Reaction as Per General Polymerization Procedure

The macromonomer from Example 5 is prepared in analogy to version 4 with the difference that, instead of 500 g of Genapol® LA-070, 500 g of Genapol® T-080 are used.

| Reactant | Amount (g) |
|---|---|
| Macromonomer | 18 |
| of version 4 - type Genapol ® T-080 | |
| NH₃-neutralized acryloyldimethyltaurine | 80 |
| tert-Butanol | 300 |
| DLP (initiator) | 2 |

### APPLICATION AND FORMULATION EXAMPLES

### Aqueous refreshing gels

### Examples 6 to 28

### General Mode of Preparation for Refreshing Gels

### Refreshing Gel

### Composition

### Example 7

| | | |
|---|---|---|
| A | Carbopol ® 980 | 0.50% |
| | Noncrosslinked copolymer with | 0.50% |
| | Laureth-7 MA as per Example 3 | |
| | Water | ad 100 |
| | NIPA ® BIOPURE 100 | 0.30% |
| | NaOH (10% in water) | 1.60% |

Preparation

| | |
|---|---|
| I | Dissolution of components A |

The formulations 6, and 8 to 28 were prepared in analogy to the general mode of preparation for refreshing gels (see Tables 2a and 2b).

TABLE 2a
Refreshing gels

| | experiment number | | | | | | |
|---|---|---|---|---|---|---|---|
| | Comparative | Inventive | Inventive | Inventive | Inventive | Inventive | Inventive |
| | Example | Example | Example | Example | Example | Example | Example |
| Ingredients | 6 | 7 | 8 | 9 | 10 | 11 | 12 |
| Carbopol ® | 1 | 0.5 | | | | | |
| 980 | | | | | | | |
| Carbopol ® | | | 0.5 | | | | |
| ETD 2020 | | | | | | | |
| Aristoflex ® | | | | 0.3 | 0.7 | | |
| AVC | | | | | | | |
| Crosslinked | | | | | | 0.7 | |
| copolymer as | | | | | | | |
| per Example | | | | | | | |
| C from DE 10 | | | | | | | |
| 2004 050239 | | | | | | | |
| Aristoflex ® | | | | | | | 0.7 |
| HMB | | | | | | | |
| Crosslinked | | | | | | | |
| copolymer as | | | | | | | |
| per Example | | | | | | | |
| 44 from EP | | | | | | | |
| 1069142 | | | | | | | |
| Crosslinked | | | | | | | |
| copolymer as | | | | | | | |
| per Example 1 | | | | | | | |
| from | | | | | | | |
| DE19625810 | | | | | | | |
| Noncrosslinked | | 0.5 | 0.5 | 0.7 | 0.3 | 0.3 | 0.3 |
| copolymer | | | | | | | |
| as per | | | | | | | |
| Example 3 | | | | | | | |
| Noncrosslinked | | | | | | | |
| copolymer | | | | | | | |
| as per | | | | | | | |
| Example 5 | | | | | | | |
| Water | 95.5 | 97.05 | 97.1 | 98.7 | 98.7 | 98.7 | 98.7 |
| NaOH | 3.2 | 1.6 | 1.6 | | | | |
| w = 10% | | | | | | | |
| Nipa | 0.3 | 0.3 | 0.3 | 0.3 | 0.3 | 0.3 | 0.3 |
| Biopure ® 100 | | | | | | | |
| NaCl | | | | | | | |
| Cirebelle ® | | | | approx. | | | |
| wax | | | | 0.05 | | | |
| Viscosity in | 50 000 | 15 800 | 14 200 | 1050 | 8850 | 8900 | 8950 |
| mPas | | | | | | | |
| (at 20 rpm) | | | | | | | |
| Appearance/ | Clear gel, | pleasantly | pleasantly | pleasantly | pleasantly | pleasantly | pleasantly |
| remarks | thick, | workable | workable | workable | workable | workable | workable |
| | stringy, | gel, not | gel, not | gel, not | gel, not | gel, not | gel, not |
| | unpleasantly | sticky | sticky | sticky | sticky | sticky | sticky |
| | sticky | | | | | | |

| | experiment number | | | | | | |
|---|---|---|---|---|---|---|---|
| | Inventive | Inventive | Comparative | Comparative | Inventive | Inventive | Comparative |
| | Example | Example | Example | Example | Example | Example | Example |
| Ingredients | 13 | 14 | 15 | 16 | 17 | 18 | 19 |
| Carbopol ® | | | 0.5 | | | | |
| 980 | | | | | | | |
| Carbopol ® | | | | | | | |
| ETD 2020 | | | | | | | |
| Aristoflex ® | 0.2 | | | | 0.5 | | |
| AVC | | | | | | | |
| Crosslinked | | | | | | | |
| copolymer as | | | | | | | |
| per Example | | | | | | | |
| C from DE 10 | | | | | | | |
| 2004 050239 | | | | | | | |
| Aristoflex ® | | | | | | 0.5 | |
| HMB | | | | | | | |
| Crosslinked | 0.5 | | | | | | |
| copolymer as | | | | | | | |
| per Example | | | | | | | |
| 44 from EP | | | | | | | |
| 1069142 | | | | | | | |
| Crosslinked | | 0.7 | | | | | |
| copolymer as | | | | | | | |
| per Example 1 | | | | | | | |
| from | | | | | | | |
| DE19625810 | | | | | | | |
| Noncrosslinked | 0.3 | 0.3 | | 1 | | | |
| copolymer | | | | | | | |
| as per | | | | | | | |
| Example 3 | | | | | | | |
| Noncrosslinked | | | | | 0.5 | 0.5 | 1 |
| copolymer | | | | | | | |
| as per | | | | | | | |
| Example 5 | | | | | | | |
| Water | 98.7 | 98.7 | 97.6 | 98.7 | 97.7 | 97.7 | 98.7 |
| NaOH | | | 1.6 | | | | |
| w = 10% | | | | | | | |
| Nipa | 0.3 | 0.3 | 0.3 | 0.3 | 0.3 | 0.3 | 0.3 |
| Biopure ® 100 | | | | | | | |
| NaCl | | | | | | | |
| Cirebelle ® | | | | | | | |
| wax | | | | | | | |
| Viscosity in | 8980 | 8950 | 26 600 | 1000 | 9750 | 9400 | 5100 |
| mPas | | | | | | | |
| (at 20 rpm) | | | | | | | |
| Appearance/ | pleasantly | pleasantly | few air | very | clear, | clear, | clear, |
| remarks | workable | workable | bubbles, | elastic gel | workable | workable | workable |
| | gel, not | gel, not | good gel | forms | gel, | gel, | gel, gel |
| | sticky | sticky | texture but | strings, | breaks up | breaks up | structure is |
| | | | sticky | relatively | quickly on | quickly on | relatively |
| | | | | liquid | the skin, | the skin, | long lasting, |
| | | | | | refreshing | refreshing | forms some |
| | | | | | | | strings |

TABLE 2b
Refreshing gels, continuation

| | experiment number | | | | |
|---|---|---|---|---|---|
| | Inventive | Inventive | Comparative | Inventive | Comparative |
| | Example | Example | Example | Example | Example |
| Ingredients | 20 | 21 | 22 | 23 | 24 |
| Carbopol ® 980 | | | | | |
| Carbopol ® ETD 2020 | | | | | |
| Aristoflex ® AVC | 0.5 | 0.5 | | | |
| Aristoflex ® HMB | | | | | 1 |
| Noncrosslinked | 2 | 2 | | 0.5 | |
| copolymer as per | | | | | |
| Example 3 | | | | | |
| Noncrosslinked | | | | | |
| copolymer as per | | | | | |
| Example 5 | | | | | |
| Water | ad 100 | ad 100 | ad 100 | ad 100 | ad 100 |
| NaOH w = 10% | | | | | |
| Nipa Biopure ® 100 | | | 0.3 | 0.3 | 0.3 |
| NaCl | | | | | 0.1 |
| Propylene glycol | 2 | 2 | | | |
| Laureth-23 | 2 | 1.8 | | | |
| Fragrance | 0.2 | | | | |
| EtOH | | 20 | | | |
| Nipaguard ® MPA | 0.5 | | | | |
| Tylose ® H 10000 G4 | | | 1 | 0.5 | |
| cirebelle ® wax | | | | | |
| Viscosity in mPas | 7500 | 1230 | 700 | 350 | 4800 |
| (at 20 rpm) | | | | | |
| Appearance/remarks | elastic gel, | workable gel, | relatively | relatively | clear gel, |
| | pleasant gel | less elastic | liquid, soft | liquid, | numerous air |
| | texture on the | than 20 | feel, slightly | pleasant gel | bubbles, |
| | skin | | sticky | texture is long | breaks up |
| | | | | lasting | very quickly |
| | | | | breaks up a | on the skin |
| | | | | bit more | |
| | | | | quickly on the | |
| | | | | skin than 25 | |

| | experiment number | | | |
|---|---|---|---|---|
| | Comparative | Comparative | Inventive | Inventive |
| | Example | Example | Example | Example |
| Ingredients | 25 | 26 | 27 | 28 |
| Carbopol ® 980 | | 1 | 0.5 | |
| Carbopol ® ETD 2020 | | | | |
| Aristoflex ® AVC | | | | |
| Aristoflex ® HMB | | | | 0.5 |
| Noncrosslinked | | | 0.5 | 0.5 |
| copolymer as per | | | | |
| Example 3 | | | | |
| Noncrosslinked | | | | |
| copolymer as per | | | | |
| Example 5 | | | | |
| Water | ad 100 | ad 100 | ad 100 | ad 100 |
| NaOH w = 10% | | 3.2 | 1.6 | |
| Nipa Biopure ® 100 | 0.3 | 0.3 | 0.3 | 0.3 |
| NaCl | 0.1 | 0.1 | 0.1 | 0.1 |
| Propylene glycol | | | | |
| Laureth-23 | | | | |
| Fragrance | | | | |
| EtOH | | | | |
| Nipaguard ® MPA | | | | |
| Tylose ® H 10000 G4 | 1 | | | |
| cirebelle ® wax | | | | |
| Viscosity in mPas | 760 | 39400 | 12200 | 1100 |
| (at 20 rpm) | | | | |
| Appearance/remarks | relatively liquid, | workable gel, | workable | some air |
| | soft fell, slightly | sticky in | gel, | bubbles, |
| | sticky | comparison to | numerous | breaks up |
| | | 27 | air | relatively |
| | | | bubbles, | quickly on the |
| | | | clear | skin |

### Hairstyling Gels

### Examples 29 to 51

### General Mode of Preparation for Hairstyling Gels:

### Hair gel, sprayable

### Composition

### Example 30

| | | |
|---|---|---|
| A | Aristoflex ® AVC | 0.15% |
| | Noncrosslinked copolymer with | 0.35% |
| | Laureth-7 MA as per Example 3 | |
| | Water | ad 100 |
| | NIPA ® BIOPURE 100 | 0.30% |
| B | Diaformer ® Z-651 N | 4.00% |

Preparation

| | |
|---|---|
| I | Dissolution of components A |
| II | Addition of B to I. |

Formulations 29 and 31 to 51 were prepared in analogy to the general mode of preparation for hairstyling gels (see Tables 3a and 3b).

TABLE 3a
Hairstyling gels

| | experiment number | | | | | | | |
|---|---|---|---|---|---|---|---|---|
| | Inventive | Inventive | Inventive | Comparative | Comparative | Comparative | Comparative | Inventive |
| | Example | Example | Example | Example | Example | Example | Example | Example |
| Ingredients | 29 | 30 | 31 | 32 | 33 | 34 | 35 | 36 |
| Aristoflex ® AVC | 0.15 | | | | 0.65 | 0.65 | | 0.35 |
| Crosslinked | | 0.15 | | | | | | |
| acryloyldimethyltaurine | | | | | | | | |
| copolymer as per Ex. C | | | | | | | | |
| from DE 10 2004 050239 | | | | | | | | |
| Aristoflex ® HMB | | | 0.15 | 0.5 | | | | |
| Noncrosslinked | 0.35 | 0.35 | 0.35 | | | | 0.5 | |
| copolymer as per Ex. 3 | | | | | | | | |
| Noncrosslinked | | | | | | | | 0.15 |
| copolymer as per Ex. 5 | | | | | | | | |
| Water | 95.2 | 95.2 | 95.2 | 95.2 | 96.05 | 95.05 | 95.2 | 96.2 |
| NIPA Biopure ® 100 | 0.3 | 0.3 | 0.3 | 0.3 | 0.3 | 0.3 | 0.3 | 0.3 |
| Eusolex ® 232 neutralized | | | | | | | | |
| Diaformer ® Z 651 N | 4 | 4 | 4 | 4 | | 4 | 4 | |
| Alkylpolyglycoside | | | | | | | | |
| (Plantacare ® 818 up) | | | | | | | | |
| Luviskol ® PVP K 30 | | | | | 3 | | | |
| VP/VA copolymer | | | | | | | | 3 |
| Diaformer ® Z 712 N | | | | | | | | |
| Propylene glycol | | | | | | | | |
| Laureth23 | | | | | | | | |
| Fragrance | | | | | | | | |
| EtOH | | | | | | | | |
| Nipaguard ® MPA | | | | | | | | |
| Viscosity in mPas | 7750 | 7800 | 10700 | 8900 | 21700 | 20600 | 7300 | 3650 |
| (at 20 rpm) | | | | | | | | |
| Appearance/remarks | elastic | elastic | elastic | gel, | yellow gel | rapid loss of | highly | workable |
| | texture, | texture, | texture, | numerous | | gel texture, | stringing, | gel, air |
| | long- | long- | long- | air bubbles, | | more watery | very elastic | bubbles, |
| | lasting | lasting | lasting | rapid loss of | | than 30/31, | | breaks |
| | gel | gel | gel | gel texture, | | less | | up quickly on |
| | texture | texture | texture | more watery | | substance | | the skin |
| | compared | compared | compared | than 30/31 | | | | |
| | with 34 | with 34 | with 32 | | | | | |

| | experiment number | | | | | | | |
|---|---|---|---|---|---|---|---|---|
| | Inventive | Inventive | Inventive | Inventive | Inventive | Inventive | Inventive | Inventive |
| | Example | Example | Example | Example | Example | Example | Example | Example |
| Ingredients | 37 | 38 | 39 | 40 | 41 | 42 | 43 | 44 |
| Aristoflex ® AVC | | 0.1 | | 0.1 | 0.5 | 0.5 | | 0.15 |
| Crosslinked | | | | | | | | |
| acryloyldimethyltaurine | | | | | | | | |
| copolymer as per | | | | | | | | |
| Ex. C from | | | | | | | | |
| DE 10 2004 050239 | | | | | | | | |
| Aristoflex ® HMB | 0.35 | | 0.1 | | | | 0.7 | |
| Noncrosslinked | | | | 1.5 | 1 | 1 | 0.3 | 0.35 |
| copolymer as per Ex. 3 | | | | | | | | |
| Noncrosslinked | 0.15 | 1 | 1 | | | | | |
| copolymer as per Ex. 5 | | | | | | | | |
| Water | 96.2 | 70.7 | 70.7 | | | | 97.7 | 94.2 |
| NIPA Biopure ® 100 | 0.3 | | | | | | 0.3 | 0.3 |
| Eusolex ® 232 neutralized | | | | | | | 1 | 1 |
| Diaformer ® Z 651 N | | | | | | | | 4 |
| Alkylpolyglycoside | | | | | | | | |
| (Plantacare ® 818 up) | | | | | | | | |
| Luviskol ® PVP K 30 | | | | | | | | |
| VP/VA copolymer | 3 | | | | | | | |
| Diaformer ® Z 712 N | | 3.5 | 3.5 | 3.5 | 3.5 | 3.5 | | |
| Propylene glycol | | 2 | 2 | 2 | 2 | 2 | | |
| Laureth23 | | 2 | 2 | 2 | 2 | 2 | | |
| Fragrance | | 0.2 | 0.2 | 0.2 | 0.2 | 0.2 | | |
| EtOH | | 20 | 20 | | | 10 | | |
| Nipaguard ® MPA | | 0.5 | 0.5 | 0.5 | 0.5 | 0.5 | | |
| Viscosity in mPas | 2800 | 10800 | 8650 | 21700 | 25700 | 9250 | 45 | 25 |
| (at 20 rpm) | | | | | | | | |
| Appearance/remarks | workable | elastic, | elastic, | almost rubber- | rubber-elastic | workable | gel | gel |
| | gel, air | relatively | relatively | elastic gel, | gel, breaks up | gel, on the | texture | texture is |
| | bubbles, | firm gel, | firm gel, | breaks up | quickly on the | skin as well, | breaks up | long |
| | breaks up | gel | gel | quickly on the | skin | gel texture | only | lasting |
| | quickly on | texture | texture | skin | | is long | slowly | |
| | the skin | long | long | | | lasting | | |
| | | lasting | lasting | | | | | |

TABLE 3b
Hairstyling gels, continuation

| | experiment number | | | | | | |
|---|---|---|---|---|---|---|---|
| | Comparative | Inventive | Inventive | Comparative | Inventive | Inventive | Inventive |
| | Example | Example | Example | Example | Example | Example | Example |
| Ingredients | 45 | 46 | 47 | 48 | 49 | 50 | 51 |
| Aristoflex ® AVC | | 0.5 | 0.1 | | 0.5 | 0.5 | 0.1 |
| Noncrosslinked | 1.5 | 2 | 2 | 1.5 | 2 | 2 | 2 |
| copolymer as per Ex. 3 | | | | | | | |
| Water | 95.2 | 70 | 69.9 | 94.7 | 71.92 | 65.9 | 60.8 |
| NIPA Biopure ® 100 | 0.3 | | | 0.3 | | | |
| Diaformer ® Z 651 N | | | | | | | |
| VP/VA Copolymer | 3 | 3 | | 3 | 3 | 3 | |
| Diaformer ® Z 712 N | | | 3.5 | | | | 5 |
| Propylene glycol | | 2 | 2 | | 2 | 2 | 2 |
| Laureth-23 | | 2 | 2 | | | 2 | 2 |
| Fragrance | | 0.2 | 0.2 | | | 0.2 | 0.2 |
| EtOH | | 20 | 20 | | 20 | 20 | 20 |
| Nipaguard ® MPA | | 0.5 | 0.5 | | 0.5 | 0.5 | 0.5 |
| Genapol ® LA 070 | | | | | | 2 | 2 |
| Silcare ® Silicone SEA | | | | | | 1 | 1 |
| Pigment: Cloisonne | | | | | ~0.04 | | |
| Super Green ® | | | | | | | |
| Glitter (Creasparkles | | | | | ~0.04 | | |
| Metallic Silver 700 ®) | | | | | | | |
| Abil ® EM 90 (silicone | | | | | | 1 | 1 |
| emulsifier) | | | | | | | |
| Viscosity in mPas (at | 370 | 2850 | 3840 | 11700 | 1390 | 9550 | 2120 |
| 20 rpm) | | | | | | | |
| Appearance/remarks | highly fluid | gel, pleasant | firm, elastic | gel | highly fluid gel, | highly fluid | elastic gel, |
| | gel, stringing | on the skin, | gel, pleasant | | pleasant on the | gel, | pleasant on the |
| | | gel texture is | on the skin, | | skin, gel | pleasant | skin, gel texture is |
| | | long lasting | gel texture is | | texture is long | on the | long lasting, |
| | | | long lasting, | | lasting, glitter | skin, gel | shimmer effect is |
| | | | shimmer | | apparent | texture is | apparent |
| | | | effect readily | | | long | |
| | | | apparent | | | lasting | |

### Mild Cleansing Gels

### Examples 52 to 55

### General Mode of Preparation for Mild Cleansing Gels:

### Mild cleansing gel

### Composition

### Example 54

| | | |
|---|---|---|
| A | Aristoflex ® AVC | 0.30% |
| | Noncrosslinked copolymer with | 0.70% |
| | Laureth-7 MA as per Example 3 | |
| | Water | ad 100% |
| | NIPA ® BIOPURE 100 | 0.30% |
| B | Genapol ® LRO | 25.90% |
| | Genagen ® CAB 818 | 10.00% |

Preparation:

| | |
|---|---|
| I | Dissolution of components A |
| II | Mixing of components B |
| III | Addition of II to I |

Formulations 52, 53 and 55 were prepared in analogy to the general mode of preparation for mild cleansing gels (see Table 4).

TABLE 4
Mild cleansing gels

| | experiment number | | | |
|---|---|---|---|---|
| | Inventive | Inventive | Inventive | Inventive |
| | Example | Example | Example | Example |
| Ingredients | 52 | 53 | 54 | 55 |
| Aristoflex ® AVC | 0.3 | | 0.3 | |
| Aristoflex ® HMB | | 0.3 | | 0.3 |
| Noncrosslinked | 0.7 | 0.7 | 0.7 | 0.7 |
| copolymer as per Ex. 3 | | | | |
| Water | 92.7 | 92.7 | 62.8 | 62.8 |
| NIPA Biopure ® 100 | 0.3 | 0.3 | 0.3 | 0.3 |
| Alkylpolyglycoside | 6 | 6 | | |
| (Plantacare ® 818 up) | | | | |
| Genapol ® LRO | | | 25.9 | 25.9 |
| Genagen ® CAB 818 | | | 10 | 10 |
| Viscosity in mPas (at | 2880 | 1615 | 250 | 1560 |
| 20 rpm) | | | | |
| Appearance/remarks | elastic, | elastic, | gel | gel |
| | gel-like | gel-like | | |

### Sunscreen Gels

### Examples 56 to 61

### General Mode of Preparation for Sunscreen Gels:

### Sunscreen gel

### Composition

### Example 60

| | | |
|---|---|---|
| A | Eusolex ® 232 | 1.00% |
| | Water | ad 100% |
| | NIPA ® BIOPURE 100 | 0.30% |
| B | Aristoflex ® AVC | 0.70% |
| | Noncrosslinked copolymer with | 0.30% |
| | Steareth-8 MA as per Example 5 | |

Preparation

| | |
|---|---|
| I | Mixing of components A and adjustment to pH 7.3 |
| | with tris(hydroxymethyl)aminomethane |
| II | Successive addition of components B to I. |

Formulations 56 to 59 and 61 were prepared in analogy to the general mode of preparation for sunscreen gels (see Table 5).

TABLE 5
Sunscreen gels

| | experiment number | | | | | |
|---|---|---|---|---|---|---|
| | Comparative | Inventive | Inventive | Inventive | Inventive | Inventive |
| | Example | Example | Example | Example | Example | Example |
| Ingredients | 56 | 57 | 58 | 59 | 60 | 61 |
| Aristoflex ® AVC | | 0.3 | | 0.7 | 0.7 | |
| Aristoflex ® HMB | | | 0.3 | | | 0.7 |
| Noncrosslinked copolymer as | 1 | 0.7 | 0.7 | 0.3 | | |
| per Ex. 3 | | | | | | |
| Noncrosslinked copolymer as | | | | | 0.3 | 0.3 |
| per Ex. 5 | | | | | | |
| Water | 97.7 | 97.7 | 97.7 | 97.7 | 97.7 | 97.7 |
| NIPA Biopure ® 100 | 0.3 | 0.3 | 0.3 | 0.3 | 0.3 | 0.3 |
| Eusolex ® 232 neutralized | 1 | 1 | 1 | 1 | 1 | 1 |
| Viscosity in mPas (at 20 rpm) | 4350 | 2920 | 1610 | 945 | 460 | 60 |
| Appearance/remarks | stringing, | more pleasant | more pleasant | pleasant on the | pleasant on | pleasant on |
| | sticks readily | texture than 56 | texture than 56 | skin | the skin | the skin |
| | when dry | | | | | |

### Styling Creams

### Examples 62 to 72

### General Mode of Preparation for Styling Creams:

### Styling cream

### Composition

### Example 62

| | | |
|---|---|---|
| A | Mineral oil, low-viscosity | 1.00% |
| | Cetiol ® 868 | 1.00% |
| | SilCare ® 15M50 | 1.50% |
| | Hostaphat ® KL 340 D | 1.00% |
| B | Aristoflex ® AVC | 1.05% |
| | Noncrosslinked copolymer with | 0.45% |
| | Laureth-7 MA as per Example 3 | |
| C | Water | ad 100.00% |
| | Glycerol | 3.00% |
| | Phenonip ® | 0.40% |
| D | Diaformer ® Z 711 N | 3.00% |

Preparation

| | |
|---|---|
| I | Mixing of A and B |
| II | Stirred incorporation of C into I |
| III | Stirred incorporation of D into II |
| IV | Homogenizing |

Formulations 63 to 72 were prepared in analogy to the general mode of preparation for styling creams (see Table 6).

TABLE 6
Styling creams

| | experiment number | | | | | |
|---|---|---|---|---|---|---|
| | Inventive | Inventive | Inventive | Inventive | Comparative | Comparative |
| | Example | Example | Example | Example | Example | Example |
| Ingredients | 62 | 63 | 64 | 65 | 66 | 67 |
| Aristoflex ® AVC | 1.05 | 0.45 | 1.05 | 1.05 | | |
| Aristoflex ® HMB | | | | | | |
| Noncrosslinked | 0.45 | 1.05 | 0.45 | 0.45 | 1 | 1 |
| copolymer as per Ex. 3 | | | | | | |
| Water | 87.6 | 87.6 | 87.6 | 87.6 | 88.1 | 88.1 |
| VP/VA Copolymer | | | | | | 3 |
| Liquid paraffin | 1 | 1 | 1 | 1 | 1 | 1 |
| Cetiol ® 868 | 1 | 1 | 1 | 1 | 1 | 1 |
| Silcare ® 15M50 | 1.5 | 1.5 | 1.5 | 1.5 | 1.5 | 1.5 |
| Tegin ® M | | | 0.3 | 0.3 | 1 | 1 |
| Hostaphat ® KL 340 D | 1 | 1 | 0.7 | | | |
| Phenonip ® | 0.4 | 0.4 | 0.4 | 0.4 | 0.4 | 0.4 |
| Glycerol | 3 | 3 | 3 | 3 | 3 | 3 |
| Diaformer ® Z 711 N | 3 | 3 | 3 | 3 | 3 | |
| Hostacerin ® DGL | | | | 0.7 | | |
| Hostacerin ® DGI | | | | | | |
| Appearance/remarks | cream, good | pleasant | cream, | cream, | no homogeneous | no homogeneous |
| | sensorial | sensorial | pleasant | pleasant | cream, | cream, |
| | properties on | properties, | on the | on the | deposition | deposition of |
| | the skin | very | skin | skin | of solids | solids |
| | | workable | | | | |

| | experiment number | | | | |
|---|---|---|---|---|---|
| | Comparative | Comparative | Comparative | Inventive | Comparative |
| | Example | Example | Example | Example | Example |
| Ingredients | 68 | 69 | 70 | 71 | 72 |
| Aristoflex ® AVC | | | | 1.05 | |
| Aristoflex ® HMB | | | | | |
| Noncrosslinked | 2 | 1 | 1 | 1.05 | 1.5 |
| copolymer as per Ex. 3 | | | | | |
| Water | 87.1 | 88.1 | 88.1 | 87.6 | 86.6 |
| VP/VA Copolymer | 3 | | 3 | | 3 |
| Liquid paraffin | 1 | 1 | 1 | 1 | 1 |
| Cetiol ® 868 | 1 | 1 | 1 | 1 | 1 |
| Silcare ® 15M50 | 1.5 | 1.5 | 1.5 | 1.5 | 1.5 |
| Tegin ® M | 0.3 | | | | |
| Hostaphat ® KL 340 D | 0.7 | | | | |
| Phenonip ® | 0.4 | 0.4 | 0.4 | 0.4 | 0.4 |
| Glycerol | 3 | 3 | 3 | 3 | 3 |
| Diaformer ® Z 711 N | | 3 | | 3 | 3 |
| Hostacerin ® DGL | | | | | |
| Hostacerin ® DGI | | 1 | 1 | 1 | 2 |
| Appearance/remarks | elastic cream | unpleasantly | elastic | very | elastic |
| | | elastic, | cream | pleasant | cream |
| | | stringing, very | | cream, very | |
| | | thin | | good | |
| | | | | sensorial | |
| | | | | properties on | |
| | | | | the skin | |

### Skin Cream (O/W Emulsion)

### Examples 73 to 75

### General Mode of Preparation for OMw Creams:

### O/W cream

### Composition

### Example 74

| | | |
|---|---|---|
| A | Mineral oil, low-viscosity | 1.00% |
| | Cetiol ® 868 | 1.00% |
| | SilCare ® 15M50 | 1.50% |
| | Hostaphat ® KL 340 D | 1.00% |
| B | Aristoflex ® AVC | 1.05% |
| | Noncrosslinked copolymer with | 0.45% |
| | Laureth-7 MA as per Example 3 | |
| C | Water | ad 100.00% |
| | Glycerol | 3.00% |
| | Phenonip ® | 0.40% |

Preparation

| | |
|---|---|
| I | Mixing of A and B |
| II | Stirred incorporation of C into I |
| III | Homogenizing |

Formulations 73 and 75 were prepared in analogy to the general mode of preparation for O/W creams (see Table 7).

TABLE 7
O/W creams

| | experiment number | | |
|---|---|---|---|
| | Comparative | Inventive | Comparative |
| | Example | Example | Example |
| Ingredients | 73 | 74 | 75 |
| Aristoflex ® AVC | | 1.05 | |
| Noncrosslinked | 1 | 0.45 | 1.5 |
| copolymer as per | | | |
| Example 3 | | | |
| Water | 88.1 | 87.6 | 86.6 |
| Liquid paraffin | 1 | 1 | 1 |
| Cetiol ® 868 | 1 | 1 | 1 |
| Silcare Silicone ® 15M50 | 1.5 | 1.5 | 1.5 |
| Tegin ® M | 0.3 | | |
| Hostaphat ® KL 340 D | 0.7 | | |
| Phenonip ® | 0.4 | 0.4 | 0.4 |
| Glycerol | 3 | 3 | 3 |
| Hostacerin ® CGI | | | 2 |
| Appearance/remarks | product | cream, pleasant | runny, |
| | separates | on the skin, | separates |
| | overnight | stable | overnight |

### Antiaging Gels

### Examples 76 to 79

### General Mode of Preparation for Antiaging Gels:

### Antiaging gel

### Composition

### Example 77

| | | |
|---|---|---|
| A | Aristoflex ® HMB | 0.30% |
| | Noncrosslinked copolymer with | 0.70% |
| | Laureth-7 MA as per Example 3 | |
| | Water | ad 100% |
| | NIPA ® BIOPURE 100 | 0.30% |
| B | Glycolic acid, (30% in water)* | 3.33% |

Preparation

| | |
|---|---|
| I | Dissolution of components A |
| II | Addition of B to I |

*neutralized with NaOH to pH 4

Formulations 76, 78, and 79 were prepared in analogy to the general mode of preparation for antiaging gels (see Table 8).

TABLE 8
Antiaging gels

| | experiment number | | | |
|---|---|---|---|---|
| | Inventive | Inventive | Inventive | Inventive |
| | Example | Example | Example | Example |
| Ingredients | 76 | 77 | 78 | 79 |
| Aristoflex ® AVG | 0.3 | | 0.3 | |
| Aristoflex ® HMB | | 0.3 | | 0.3 |
| Noncrosslinked | 0.7 | 0.7 | | |
| copolymer as per | | | | |
| Example 3 | | | | |
| Noncrosslinked | | | 0.7 | 0.7 |
| copolymer as per | | | | |
| Example 5 | | | | |
| Water | 99.7 | 99.7 | 95.4 | 95.4 |
| NIPA Biopure ® 100 | 0.3 | 0.3 | 0.3 | 0.3 |
| Glycolic acid, neutralized | 3.33 | 3.33 | 3.33 | 3.33 |
| Viscosity in mPas | 880 | 795 | 190 | 55 |
| (at 20 rpm) | | | | |
| Appearance/remarks | stable gel | stable gel | fluid | fluid |

Chemical identification (INCI nomenclature) of commercial products used:

| | |
|---|---|
| Abil ® EM-90 | Cetyl PEG/PPG-10/1 Dimethicone |
| Aristoflex ® AVC | Ammonium Acryloyldimethyltaurate/VP |
| | Copolymer |
| Aristoflex ® HMB | Ammonium Acryloyldimethyltaurate/ |
| | Beheneth-25 Methacrylate Crosspolymer |
| Carbopol ® 980 | Carbomer |
| Cetiol ® 868 | Ethylhexyl Stearate |
| Cloisonne ® Super Green | Mica and Titanium Dioxide and Iron |
| | Oxides and Ferric |
| | Ferrocyanide |
| Creasparkles Metallic | Polyethylene Terephthalate and Glycerol and |
| Silver 700 ® (Glitter) | Acrylamide/Ammonium Acrylate Copolymer |
| | and Aluminum Powder |
| Diaformer ® Z-651 N | Acrylates/Lauryl Acrylate/Stearyl Acrylate/ |
| | Ethylamine Oxide Methacrylate Copolymer |
| Diaformer ® Z 711 N | Acrylates/Lauryl Acrylate/Stearyl Acrylate/ |
| | Ethylamine Oxide Methacrylate Copolymer |
| Diaformer ® Z 712 N | Acrylates/Lauryl Acrylate/Stearyl Acrylate/ |
| | Ethylamine Oxide Methacrylate Copolymer |
| Eusolex ® 232 | Phenylbenzimidazole Sulfonic Acid |
| Genagen ® CAB 818 | Cocamidopropyl Betaine |
| Genapol ® LA 070 | Laureth-7 |
| Genapol ® LRO | Sodium Laureth Sulfate |
| Hostacerin ® DGI | Polyglyceryl-2 Sesquiisostearate |
| Hostacerin ® DGL | PEG-10 Polyglyceryl-2 Laurate |
| Hostaphat ® KL 340 D | Trilaureth-4 Phosphate |
| NIPA Biopure ® 100 | Imidazolidinyl Urea |
| Nipaguard ® MPA | Benzyl Alcohol and Methylparaben and |
| | Propylparaben |
| Phenonip ® | Phenoxyethanol and Methylparaben and |
| | Ethylparaben |
| | and Butylparaben |
| | and Propylparaben |
| | and isobutylparaben |
| Plantacare ® 818 up | Coco-Glucoside |
| Luviskol ® PVP K 30 | PVP (Polyvinylpyrrolidone) |
| SilCare ® Silicone SEA | Trideceth-9 PG-Amodimethicone and |
| | Trideceth-12 |
| SilCare ® 15M50 | Phenyltrimethicone |
| Tegin ® M | Glyceryl Stearate |
| Tylose ® H 10000 G4 | Hydroxyethylcellulose |

1. A cosmetic or pharmaceutical preparation comprising:
I) at least one water-soluble non-crosslinked copolymer including
A) at least one structural unit of the formula (1) 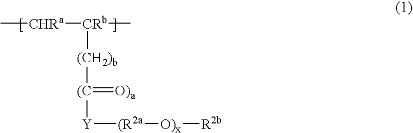
wherein
R^{a} is H or CH₃;
R^{b} is H or CH₃;
a is 0 or 1;
b is 0 or 1;
Y is O, S, PH or NH;
R^{2a} is a linear or branched (C₂-C₄)-alkylene group;
x is an integer between 1 and 500; and
R^{2b} is hydrogen or a saturated or mono- or polyunsaturated linear or branched aliphatic, cycloaliphatic or aromatic (C₁-C₃₀)-hydrocarbon radical, and
B) at least one structural unit of the formula (2) 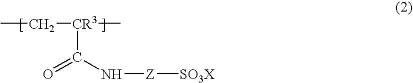
wherein
R³ is hydrogen, methyl or ethyl,
Z is (C₁-C₈)-alkylene, and
X is singly to triply ethoxylated ammonium compounds having the same degree or different degrees of ethoxylation, hydrogen, lithium, sodium, potassium, magnesium, calcium, ammonium, monoalkylammonium, dialkylammonium, trialkylammonium or tetraalkylammonium, the alkyl substituents of the ammonium ions being independently of one another (C₁-C₂₂)-alkyl radicals optionally substituted by 0 to 3 hydroxyalkyl groups with an alkyl chain length in a range from C₂ to C₁₀, or a mixture thereof and
II) at least one water-soluble or water-swellable crosslinked or non-crosslinked copolymeric or homopolymeric thickener.

2. The preparation according to claim 1, wherein R^{a}, R^{b}, a, and b in the structural unit of the formula (1) are selected from the group consisting of the following combinations:
R^{a}=R^{b}=H and a=b=0;
R^{a}=R^{b}=H, a=0 and b=1;
R^{a}=R^{b}=H, a=1 and b=0; and
R^{a}=H, R^{b}=CH₃, a=1 and b 0.

3. The preparation according to claim 2, wherein R^{a}, R^{b}, a, and b in the structural unit of the formula (1) are selected from the group consisting of the following combinations:
R^{a}=R^{b}=H, a=1 and b=0 and
R^{a}=H, R^{b}=CH₃, a=1 and b=0.

4. The preparation according to claim 1, wherein, in the structural unit of the formula (1),
R^{2a} is an ethylene or propylene radical,
x is a number between 3 and 50, and
R^{2b} is a saturated or a mono- or polyunsaturated aliphatic or cycloaliphatic hydrocarbon radical.

5. The preparation according to claim 1, wherein R^{2b} in the structural unit of the formula (1) is a (C₆-C₂₂)-hydrocarbon radical.

6. The preparation according to claim 5, wherein the hydrocarbon radical is an alkyl or a mono- or polyunsaturated alkenyl radical.

7. The preparation according to claim 1, wherein R^{2b} in the structural unit of the formula (1) is a radical selected from the group consisting of: stearyl, lauryl, cocoyl, undecyl, behenyl, cetearyl, cetyl, and myristyl.

8. The preparation according to claim 1, wherein, in the structural unit of the formula (2), R³ is H, Z is —C(CH₃)₂—CH₂—, and X is hydrogen, sodium, potassium, ammonium, or a mixture thereof.

9. The preparation according to claim 1, wherein the degree of neutralization of the structural unit of the formula (2) is 70 to 100 mol %.

10. The preparation according to claim 1, wherein the molar fractions of the structural unit of the formula (1) and of the structural unit of the formula (2) in the copolymer of component I) are in each case from 0.1 to 99.9 mol %.

11. The preparation according to claim 1, wherein the fraction of the structural unit of the formula (1) in the copolymer of component I) is from 50.1 to 99.9 mol %.

12. The preparation according to claim 1, wherein the fraction of the structural unit of the formula (1) in the copolymer of component I) is from 0.1 to 50 mol %.

13. The preparation according to claim 1, wherein the at least one water-soluble or water-swellable crosslinked or non-crosslinked copolymeric or homopolymeric thickener of component II) is selected from the group consisting of:
a) a polymer based on methacrylic acid or acrylic acid and modified (meth)acrylic acid,
b) a homopolymer of dimethylaminoethyl(meth)acrylates, quaternized with methyl chloride,
c) a copolymer of dimethylaminoethyl(meth)acrylate, quaternized with methyl chloride and acrylamide,
d) a crosslinked copolymer of vinyl isodecanoate and (meth)acrylic acid,
e) a polyvinyl alcohol,
f) a polyvinyl methyl ether,
g) a polyacrylamide,
h) a polyvinylamide,
i) a polyvinylpyrrolidone,
j) a poly(meth)acrylic acid, a poly(meth)acrylic ester, and other poly(meth)acrylic acid derivatives,
k) a polyethylene oxide,
l) a copolymer of maleic anhydride and vinyl methyl ether,
m) a polysulfonic acid,
n) a crosslinked homopolymer of acrylamidoalkylsulfonic acid, a salt thereof, or a mixture thereof,
o) copolymers of acrylamidoalkylsulfonic acid, a salt thereof, or a mixture thereof, and comonomers selected from the group consisting of: acrylamide, hydroxyethyl(meth)acrylate and cationically modified (meth)acrylates, and
p) a natural and modified natural polymer based on a polysaccharide.

14. The preparation according to claim 1, wherein the at least one water-soluble or water-swellable crosslinked or non-crosslinked copolymeric or homopolymeric thickener of component II) is selected from the group consisting of:
a) a polymer based on methacrylic acid or acrylic acid and modified (meth)acrylic acid,
g) a polyacrylamide,
j) a poly(meth)acrylic acid, a poly(meth)acrylic ester, and other poly(meth)acrylic acid derivatives,
m) a polysulfonic acid,
n) a crosslinked homopolymer of acrylamidoalkylsulfonic acid, a salt thereof, or a mixture thereof,
o) a copolymer of acrylamidoalkylsulfonic acid, a salt thereof, or a mixture thereof, and comonomer selected from the group consisting of: acrylamide, hydroxyethyl (meth)acrylate and cationically modified (meth)acrylates, and
p) a natural and modified natural polymer based on a polysaccharide.

15. The preparation according to claim 1, wherein the weight fraction of crosslinking comonomers, based on the total mass of the polymers of component II), is from 0% to 20% by weight.

16. The preparation according to claim 1, wherein the at least one water-soluble or water-swellable crosslinked or non-crosslinked copolymeric or homopolymeric thickener of component II) is a copolymeric thickener selected from the group consisting of:
a1) 1% to 50% by weight of the structural repeating unit of the formula (3) 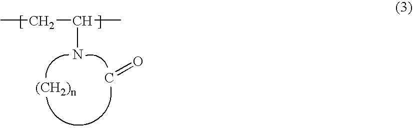
where n is an integer from 2 to 9;
a2) 1% to 50% by weight of the structural repeating unit of the formula (4) 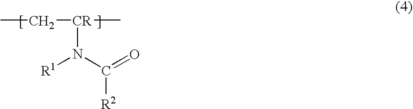
where R, R¹ and R² are identical or different and are hydrogen or a linear or branched alkyl or alkenyl group having in each case 1 to 30, carbon atoms; and
a3) 1% to 50% by weight of a mixture of the structural repeating unit of the formula (3) and the structural repeating unit of the formula (4); and
b) 49.99% to 98.99% by weight of the structural repeating unit of the formula (2) 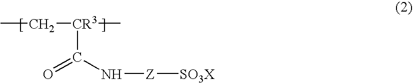
wherein
R³ is hydrogen, methyl or ethyl,
Z is (C₁-C₈)-alkylene, and
X is singly to triply ethoxylated ammonium compounds having the same degree or different degrees of ethoxylation, hydrogen, lithium, sodium, potassium, magnesium, calcium, ammonium, monoalkylammonium, dialkylammonium, trialkylammonium or tetraalkylammonium, the alkyl substituents of the ammonium ions being independently of one another (C₁-C₂₂)-alkyl radicals optionally substituted by 0 to 3 hydroxyalkyl groups with an alkyl chain length in a range from C₂ to C₁₀, or a mixture thereof, and
c) 0% to 8% by weight of crosslinking structures derived from monomers having at least two olefinic double bonds.

17. The preparation according to claim 1, wherein the at least one water-soluble or water-swellable crosslinked or non-crosslinked copolymeric or homopolymeric thickener of component II) is a crosslinked polymer comprising crosslinking structures derived from methylenebisacrylamide; methylenebismethacrylamide; esters of unsaturated monocarboxylic and polycarboxylic acids with polyols.

18. The preparation according to claim 1, wherein the at least one water-soluble or water-swellable crosslinked or noncrosslinked copolymeric or homopolymeric thickener of component II) is a crosslinked polymer comprising crosslinking structures derived from trimethylolpropane triacrylate.

19. The preparation according to claim 1, wherein the at least one water-soluble non-crosslinked copolymers of component I) the at least one water-soluble or water-swellable crosslinked or non-crosslinked copolymeric or homopolymeric thickener of component II) or both contain one or more further structural units derived from at least one monomer selected from olefinically unsaturated acids and their salts with monovalent and divalent counterions.

20. The preparation according to claim 1, wherein the weight ratio of the at least one water-swellable non-crosslinked polymer of component I), to the at least one water-soluble or water-swellable polymeric thickener of component II) is in the range from 1 to 99:99 to 1.

21. The preparation according to claim 1, containing the polymer mixture of components I) and II) in an amount of from 0.1% to 10% by weight.

22. The preparation according to claim 1, wherein the cosmetic or pharmaceutical preparation is in the form of a hair treatment, haircare, hairstyling or hair cleaning composition.

23. The preparation according to claim 1, wherein the cosmetic or pharmaceutical preparation is in the form of an aqueous, gel-like cosmetic or pharmaceutical composition.

24. The preparation according to claim 1, wherein the cosmetic or pharmaceutical preparation is in the form of a hair gel.

25. The preparation according to claim 1, wherein the cosmetic or pharmaceutical preparation is in a sprayable form.

26. The preparation according to claim 1, comprising one or more film formers.

27. The preparation according to claim 1, comprising one or more UV filters.

28. The preparation according to claim 1, comprising one or more antioxidants.

29. The preparation according to claim 1, wherein the cosmetic or pharmaceutical preparation is transparent or translucent.

30. The preparation according to claim 1, which is wherein the cosmetic or pharmaceutical preparation is emulsifier-free, oil-free, or both.

31. The preparation according to claim 1, comprising as component II) at least one water-soluble or water-swellable crosslinked copolymeric thickener containing at least one structural units of the formula (1), at least one structural units of the formula (2), and at least one crosslinking structural units derived from monomers having at least two olefinic double bonds.

32. The preparation according to claim 1, wherein R^{2b} in the structural unit of the formula (1) is a (C₁₂-C₁₈)-hydrocarbon radical.

33. The preparation according to claim 5, wherein the hydrocarbon radical is an alkyl radical.

34. The preparation according to claim 1, wherein R^{2b} in the structural unit of the formula (1) is a radical selected from the group consisting of: stearyl, lauryl, cetyl, and myristyl.

35. The preparation according to claim 1, wherein, in the structural unit of the formula (2), R³ is H, Z is —C(CH₃)₂—CH₂—, and X is hydrogen, ammonium, or a mixture thereof.

36. The preparation according to claim 1, wherein the degree of neutralization of the structural unit of the formula (2) is 80 to 100 mol %.

37. The preparation according to claim 1, wherein the degree of neutralization of the structural unit of the formula (2) is 80 to 99 mol %.

38. The preparation according to claim 1, wherein the fraction of the structural unit of the formula (1) in the copolymer of component I) is from 70 to 95 mol %.

39. The preparation according to claim 1, wherein the fraction of the structural unit of the formula (1) in the copolymer of component I) is from 80 to 90 mol %.

40. The preparation according to claim 1, wherein the fraction of the structural unit of the formula (1) in the copolymer of component I) is 5 to 25 mol %.

41. The preparation according to claim 1, wherein the fraction of the structural unit of the formula (1) in the copolymer of component I) is from 6 to 15 mol %.

42. The preparation according to claim 13, wherein the at least one water-soluble or water-swellable crosslinked or non-crosslinked copolymeric or homopolymeric thickener of component II) is:
a) a polymer derived from crosslinked polymers of acrylic acid, copolymers of (meth)acrylic acid and polyalkylene polyether, and hydrophobically modified poly(meth)acrylates.

43. The preparation according to claim 13, wherein the at least one water-soluble or water-swellable crosslinked or non-crosslinked copolymeric or homopolymeric thickener of component II) is:
m) a copolymer based on acrylamidoalkylsulfonic acid, a salt thereof, or a mixture thereof, and one or more comonomers selected from cyclic N-vinylcarboxamides and linear N-vinylcarboxamides, or hydrophobically modified crosslinked acrylamidoalkylsulfonic acid copolymers.

44. The preparation according to claim 13, wherein the at least one water-soluble or water-swellable crosslinked or non-crosslinked copolymeric or homopolymeric thickener of component II) is:
p) a cellulose ether, a cellulose derivative, carboxymethylcellulose, hydroxyethylcellulose, a gelatin, a starch, a starch derviative, a sodium alginate, a xanthan, a guar, a guar derivative, scleroglucan, tragacanth or a dextrin derivative.

45. The preparation according to claim 13, wherein the at least one water-soluble or water-swellable crosslinked or non-crosslinked copolymeric or homopolymeric thickener of component II) is:
p) a dextrin ester.

46. The preparation according to claim 17, wherein the at least one water-soluble or water-swellable crosslinked or non-crosslinked copolymeric or homopolymeric thickener of component II) is a crosslinked polymer comprising crosslinking structures derived from butanediol and ethylene glycol diacrylate and methacrylate, trimethylolpropane triacrylate (TMPTA) and trimethylolpropane trimethacrylate (TMPTMA); an allyl compound, an-allyl ester of phosphoric acid; and/or a vinylphosphonic acid derivative or a mixture thereof.

47. The preparation according to claim 17, wherein the at least one water-soluble or water-swellable crosslinked or non-crosslinked copolymeric or homopolymeric thickener component II) is a crosslinked polymer comprising crosslinking structures originating derived from allyl(meth)acrylate, triallyl cyanurate, diallyl maleate, polyallyl esters, tetraallyloxyethane, triallylamine, tetraallylethylenediamine; and/or trimethylolpropane triacrylate (TMPTA) or a mixture thereof.

48. The preparation according to claim 19, wherein the at least one water-soluble non-crosslinked copolymer of component I) the at least one water-soluble or water-swellable crosslinked or non-crosslinked copolymeric or homopolymeric thickener of component II) or both contain at least one further structural unit derived from N-vinylformamide (NVF), N-vinylmethylformamide, N-vinylmethylacetamide (VIMA), N-vinylacetamide, N-vinylpyrrolidone (NVP), N-vinylcaprolactam; or amides of acrylic or of methacrylic acid.

49. The preparation according to claim 19, wherein the at least one water-soluble non-crosslinked copolymer of component I) and/or the at least one water-soluble or water-swellable crosslinked or non-crosslinked copolymeric or homopolymeric thickener of component II) or both contain at least one further structural unit derived from acrylamide, N,N-dimethylacrylamide, N,N-diethylacrylamide, alkoxylated acrylamides and methacrylamides.

50. The preparation according to claim 20, wherein the weight ratio of the non-crosslinked copolymer of component I), to the at least one water-soluble or water-swellable polymeric thickener of component II) is in the range from 1 to 99:99 to 1.

51. The preparation according to claim 20, wherein the weight ratio of the at least one water-swellable non-crosslinked polymer of component I), to the at least one water-soluble or water-swellable polymeric thickener of component II) is in the range from 10 to 90:90 to 10.

52. The preparation according to claim 20, wherein the weight ratio of the at least one water-swellable non-crosslinked polymer of component I), to the at least one water-soluble or water-swellable polymeric thickener of component II) is in the range from 20 to 80:80 to 20.

53. The preparation according to claim 20, wherein the weight ratio of the at least one water-swellable non-crosslinked polymer of component I), to the at least one water-soluble or water-swellable polymeric thickener of component II) is in the range from 30 to 70:70 to 30.

54. The preparation according to claim 1, wherein the cosmetic or pharmaceutical preparation is in the form of a transparent or translucent, colorless hair gel.

55. The preparation according to claim 4, wherein, in the structural unit of the formula (1),
R^{2a} is an ethylene radical.

56. The preparation according to claim 4, wherein, in the structural unit of the formula (1),
x is a number between 6 and 30.

57. The preparation according to claim 16, wherein R, R¹ and R² are identical or different and are hydrogen or a linear or branched alkyl or alkenyl group having in each case 1 to 20 carbon atoms.

58. The preparation according to claim 16, wherein R, R¹ and R² are identical or different and are hydrogen or a linear or branched alkyl or alkenyl group having in each case 1 to 12 carbon atoms.

59. The preparation according to claim 17, wherein the at least one water-soluble or water-swellable crosslinked or non-crosslinked copolymeric or homopolymeric thickener or of component II) is a crosslinked polymer comprising crosslinking structures derived from di-acrylates and tri-acrylates and -methacrylates.

60. The preparation according to claim 19, wherein the at least one or more water-soluble non-crosslinked copolymers of component I) and/or the at least one water-soluble or water-swellable crosslinked or non-crosslinked copolymeric or homopolymeric thickener of component II) or both contain one or more further structural units derived from styrenesulfonic acid, vinylsulfonic acid, vinylphosphonic acid, allylsulfonic acid, methallylsulfonic acid, acrylic acid, (meth)acrylic acid, maleic acid and maleic anhydride and salts thereof; esters of (meth)acrylic acid with aliphatic, aromatic or cycloaliphatic alcohols having a carbon number from 1 to 22; esters of (meth)acrylic acid with alkyl ethoxylates, open-chain and cyclic N-vinyl amides (N-vinyllactams) having a ring size of 4 to 9 atoms.

61. The preparation according to claim 19, wherein the at least one water-soluble non-crosslinked copolymer of component I) and/or the at least one water-soluble or water-swellable crosslinked or non-crosslinked copolymeric or homopolymeric thickener of component II) or both contain at least one further structural unit derived from MAPTAC and APTAC; 2-vinylpyridine; 4-vinylpyridine; vinyl acetate; glycidyl methacrylate; acrylonitrile; vinyl chloride; vinylidene chloride; tetrafluoroethylene and/or DADMAC.
